(19) United States
(12) Patent Application Publication
(10) Pub. No.: US 2023/0149732 A1
(43) Pub. Date: May 18, 2023

(54) COMBINED NITRITE AND LIGHT TREATMENT TO PREVENT DEVICE THROMBOSIS

(71) Applicant: Wake Forest University Health Sciences, Winston-Salem, NC (US)

(72) Inventors: Daniel B. Kim-Shapiro, Winston-Salem, NC (US); James Eric Jordan, Winston-Salem, NC (US); Elmira Alipour, Winston-Salem, NC (US); Nadeem Wajih, Winston-Salem, NC (US)

(21) Appl. No.: 17/989,669

(22) Filed: Nov. 17, 2022

Related U.S. Application Data
(60) Provisional application No. 63/280,459, filed on Nov. 17, 2021.

(51) Int. Cl. A61N 5/06

(52) U.S. Cl. CPC A61N 5/062; A61N 2005/0663; A61N 2005/0651

(57) ABSTRACT

A method for inhibiting device thrombosis for a patient may include circulating blood through an extracorporeal circuit coupled to the patient, infusing nitrite into the flow of blood such that the nitrite is bioactivated by red blood cells to form nitric oxide, and illuminating the flow of blood with far-red light to increase bioactivation of the nitrite by the red blood cells. A system for inhibiting device thrombosis for a patient may include an extracorporeal circuit configured to circulate a flow of blood, a nitrite infusion device coupled to the extracorporeal circuit and configured to infuse nitrite into the flow of blood, and one or more light sources disposed along the extracorporeal circuit and configured to illuminate the flow of blood with far-red light.

This application claims the benefit of U.S. Provisional Application No. 63/280,459, filed on Nov. 17, 2021, which is incorporated herein by reference in its entirety.

### FIELD OF THE DISCLOSURE

The present disclosure relates generally to techniques for preventing thrombosis and more particularly to systems and methods for combined nitrite and light treatment to prevent device thrombosis.

### BACKGROUND OF THE DISCLOSURE

Device thrombosis is a significant complication in otherwise life-saving interventions including hemodialysis catheters, stents, extracorporeal devices, and in left and right ventricular assist devices. Standard care often involves use of a systemic anti-coagulant but this can lead to bleeding and, in some cases, heparin-induced thrombocytopenia. Improved systems and methods to locally reduce thrombosis would be advantageous.

### SUMMARY OF THE DISCLOSURE

The present disclosure provides methods and systems for inhibiting device thrombosis for a patient. In one aspect, a method for inhibiting device thrombosis for a patient is provided. In one aspect, the method may include circulating blood through an extracorporeal circuit coupled to the patient, infusing nitrite into the flow of blood such that the nitrite is bioactivated by red blood cells to form nitric oxide, and illuminating the flow of blood with far-red light to increase bioactivation of the nitrite by the red blood cells.

In some aspects, infusing the nitrite into the flow of blood may include infusing the nitrite in line with the extracorporeal circuit. In some aspects, infusing the nitrite into the flow of blood may include continuously infusing the nitrite for a period of time while the flow of blood circulates through the extracorporeal circuit. In some aspects, illuminating the flow of blood with the far-red light may include illuminating the flow of blood with the far-red light in line with the extracorporeal circuit. In some aspects, illuminating the flow of blood with the far-red light may include continuously illuminating the flow of blood with the far-red light for a period of time while the flow of blood circulates through the extracorporeal circuit. In some aspects, illuminating the flow of blood with the far-red light may include passing the flow of blood through a section of tubing disposed between a pair of light sources. In some aspects, the section of tubing may be arranged in a coil. In some aspects, the pair of light sources may include a pair of light-emitting diodes. In some aspects, illuminating the flow of blood with the far-red light may include illuminating the flow of blood with the far-red light emitted at a wavelength of 660 nm. In some aspects, the method also may include maintaining the flow of blood circulating through the extracorporeal circuit at a predetermined temperature using a circulating water bath.

Also disclosed is a system for inhibiting device thrombosis for a patient is provided. In one aspect, the system may include an extracorporeal circuit configured to circulate a flow of blood of the patient, a nitrite infusion device coupled to the extracorporeal circuit and configured to infuse nitrite into the flow of blood such that the nitrite is bioactivated by red blood cells to form nitric oxide, and one or more light sources disposed along the extracorporeal circuit and configured to illuminate the flow of blood with far-red light to increase bioactivation of the nitrite by the red blood cells.

In some aspects, the nitrite infusion device may be configured to infuse the nitrite in line with the extracorporeal circuit. In some aspects, the nitrite infusion device may be configured to infuse the nitrite for a period of time while the flow of blood circulates through the extracorporeal circuit. In some aspects, the one or more light sources may be configured to illuminate the flow of blood with the far-red light in line with the extracorporeal circuit. In some aspects, the one or more light sources may be configured to illuminate the flow of blood with the far-red light for a period of time while the flow of blood circulates through the extracorporeal circuit. In some aspects, the extracorporeal circuit may include a section of tubing disposed adjacent to the one or more light sources. In some aspects, the section of tubing may be arranged in a coil. In some aspects, the one or more light sources may include a pair of light sources, and the section of tubing may be disposed between the pair of light sources. In some aspects, the pair of light sources may include a pair of light-emitting diodes. In some aspects, the one or more light sources may be configured to emit the far-red light at a wavelength of 660 nm. In some aspects, the system also may include a circulating water bath configured to maintain the flow of blood circulating through the extracorporeal circuit at a predetermined temperature.

These and other aspects and improvements of the present disclosure will become apparent to one of ordinary skill in the art upon review of the following detailed description when taken in conjunction with the several drawings and the appended claims.

### BRIEF DESCRIPTION OF THE DRAWINGS

FIG. 1 illustrates nitrate increasing nitric oxide bioavailability through a mechanism involving red blood cell bioactivation and this action being potentiated by far-red light illumination that can be used for treatments aimed at decreasing thrombosis.

FIG. 2 illustrates red blood cell bioactivation of nitrite being enhanced by far-red light.

FIG. 3 illustrates nitrite mediated inhibition of platelet activation involving nitric oxide and red blood cell surface thiols.

FIG. 4 illustrates catalysis of the reaction of nitric oxide and heme by thiols.

FIG. 5 illustrates red blood cell thiol nitrosation inhibited platelet activation.

FIG. 6 illustrates heme-nitric-oxide export.

FIG. 7 illustrates platelet adhesion under flow conditions.

FIG. 8 illustrates the effect of far-red light on a heme-nitrosyl mixture in the presence of thiols.

FIG. 9 schematically illustrates an example system for nitrite and light treatment to inhibit device thrombosis.

FIG. 10 illustrates an example system for nitrite and light treatment to inhibit device thrombosis.

FIG. 11 illustrates pressure data and circuit survival data collected using the system of FIG. 10.

FIG. 12 illustrates Kaplan-Meier circuit survival curves of data collected using the system of FIG. 10.

FIG. 13 illustrates pressure data and circuit survival data collected using the system of FIG. 10.

FIG. 14 illustrates pressure data and circuit survival data collected using the system of FIG. 10.

FIG. 15 illustrates platelet count data collected using the system of FIG. 10.

FIG. 16 illustrates pressure data and circuit survival data collected using the system of FIG. 10.

FIG. 17 illustrates Kaplan-Meier circuit survival curves of data collected using the system of FIG. 10.

FIG. 18 illustrates Kaplan-Meier circuit survival curves of data collected using the system of FIG. 10.

FIG. 19 illustrates circuit survival improvement data collected using the system of FIG. 10.

The detailed description is set forth with reference to the accompanying drawings. The drawings are provided for purposes of illustration only and merely depict example embodiments of the disclosure. The drawings are provided to facilitate understanding of the disclosure and shall not be deemed to limit the breadth, scope, or applicability of the disclosure. The use of the same reference numerals indicates similar, but not necessarily the same or identical components. Different reference numerals may be used to identify similar components. Various embodiments may utilize elements or components other than those illustrated in the drawings, and some elements and/or components may not be present in various embodiments. The use of singular terminology to describe a component or element may, depending on the context, encompass a plural number of such components or elements and vice versa.

### DETAILED DESCRIPTION OF THE DISCLOSURE

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, and components have not been described in detail so as not to unnecessarily obscure aspects of the embodiments.

Device thrombosis is a significant complication in otherwise life-saving interventions including hemodialysis catheters, stents, extracorporeal devices, and in left and right ventricular assist devices. Standard care often involves use of a systemic anti-coagulant but this can lead to bleeding and, in some cases, heparin-induced thrombocytopenia. Thus, methods to locally reduce thrombosis would be advantageous. Nitric oxide (NO) has potential in treating thrombotic conditions. The disclosed methods and systems are based largely on the premise that nitrite, once considered to be biologically inert in human physiology, is converted to the important signaling molecule NO by hemoglobin. It has been shown that red blood cells (RBCs) bioactivate nitrite and inhibit platelet activation through an NO-dependent mechanism. It has also been shown that nitrite is a unique NO donor that, unlike other NO donors that lose activity in the presence of RBCs, requires RBCs for bioactivation. Thus, nitrite is the best suited NO therapeutic for action in blood. It has further been shown that far-red light illumination dramatically enhances nitrite bioactivation by RBCs and enhances inhibition of platelet activation. Device thrombosis is an excellent starting point in developing techniques to treat thrombosis as limitations due to penetration depth of the light into tissue are avoided. This disclosure teaches that nitrite increases NO bioavailability through a mechanism involving RBC bioactivation and provides methods and systems through which this action is potentiated with far-red light illumination that can be used for treatment (and potentially prevention) of thrombotic disorders, particularly device thrombosis.

Nitrite is bioactivated by RBCs, producing NO activity through a function of hemoglobin (Hb),

H⁺+NO₂⁻+Hb→MetHb+NO+OH⁻, (Eq. 1)

where MetHb is the ferric, oxidized form, methemoglobin. that this reaction is allosterically controlled so that it is faster in high oxygen affinity R-state Hb than in low oxygen affinity T-state Hb making it so that it can occur at intermediate to high Hb oxygen saturations pertinent to physiological conditions. However, the reaction shown in Equation 1 was previously thought to be irrelevant physiologically. The notion that nitrite is converted to NO by Hb within the RBC is challenged by the fact that NO reacts with Hb extremely fast. NO is rapidly scavenged by oxygenated Hb (OxyHb),

NO+HbO₂→MetHb+NO₃⁻, (Eq. 2)

governed by a rate constant of 6-8×10⁷ M⁻¹s⁻¹. NO also reacts with deoxygenated Hb, binding to the heme to form iron nitrosyl Hb (HbNO), Hb+NO→HbNO. This reaction may temporarily preserve NO activity but the dissociation rate of NO from Hb is very slow. Moreover, when the NO is released from HbNO, it must now escape the 20 mM Hb (in heme) inside the RBC, which is highly unlikely.

Neither nitrite nor RBCs alone potentiate vasodilation of aortic rings, but the combination of both does. It is the Hb, rather than other RBC enzymes, that is responsible for nitrite bioactivation.

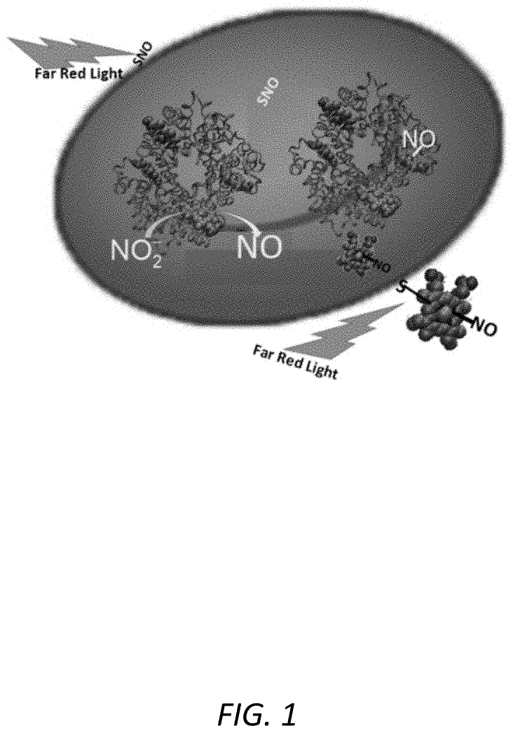

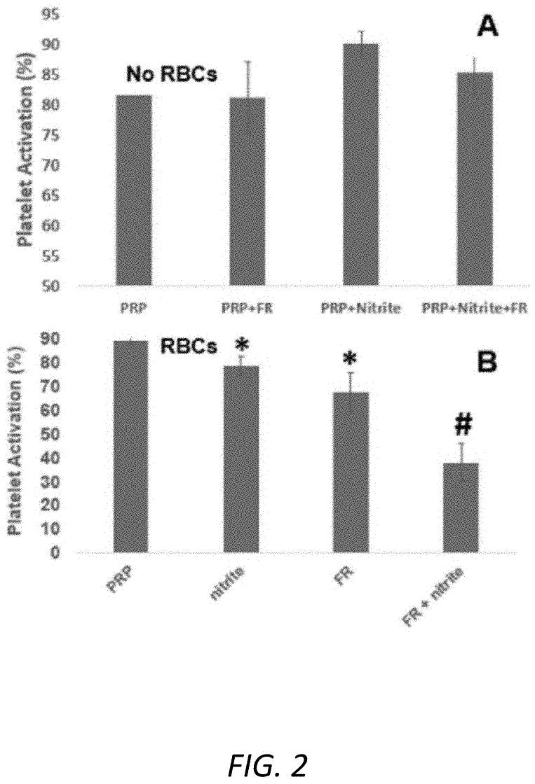

This disclosure also teaches that far-red and near infrared light dramatically enhance nitrite bioactivation by RBCs. Far-red light (about 600-750 nm) and near infrared light (about 700-1400 nm) have been used clinically for improved wound healing, tissue generation, reduction of inflammation, and increasing cerebral blood flow to improve cognition. The mechanism for these actions has been attributed generally to increased blood flow, tissue oxygenation, and angiogenesis secondary to improved mitochondrial function attributed to cytochrome c oxidase redox state and function. However, it was not clear how improvement in cytochrome c oxidation state could account for increased blood flow and other effects of infrared/far-red light. This disclosure teaches that RBC bioactivation is enhanced by exposure to far-red and near infrared light, indicating that a NO-containing species exists on RBCs (FIG. 1) that is photolabile. The data shown in FIG. 2 strongly supports this premise (note scales). Previous work using inhibition of platelet activation as a measure of nitrite bioactivation has shown that (i) RBCs are necessary for nitrite-mediated inhibition of platelet activation (while other NO donors' ability to inhibit platelet activation is blunted by the presence of RBCs), (ii) this action is abrogated by an NO scavenger, (iii) this action is achieved with plasma levels of nitrite easily attained through dietary nitrate interventions, and (iv) this action involves RBC surface thiols. Data in FIG. 2A show that in the absence of RBCs, short exposure (˜5 min) to low intensity far-red light (660 nm) has no effect on platelet activation, both in the presence and absence of nitrite. However, when RBCs are present, nitrite decreases platelet activation and the light dramatically enhances nitrite bioactivation by RBCs as measured by inhibition of platelet activation (FIG. 2B). Additionally, the combination of light and nitrite is more effective than either treatment alone using a variety of initiators and measures of clotting. Interestingly, far-red light significantly decreases platelet activation in the presence of deoxygenated RBCs even when not pre-treated with nitrite (FIG. 2B). This is attributed to accumulating steady state NO species on the surface of the RBCs. The effects of far-red light without nitrite treatment are small and variable, perhaps reflecting small amounts of nitrite present at steady state and varying between individuals due to diet and other factors.

As described herein, NO production and red light illumination are beneficial in treating thrombosis. Formation of a blot clot is often a beneficial process to stop bleeding and repair damage after a wound. However, formation of a blood clot when there is no wound can lead to a great degree of morbidity and mortality; this process is referred to as thrombosis. There are lots of types of thrombosis including deep vein thrombosis, arterial thrombosis, and (the focus of this disclosure) device thrombosis. Use of several devices including stents, heart valves, vascular grafts, catheters, left and right ventricular assist devices, and extracorporeal devices (including those used in cardiopulmonary bypass surgery and extracorporeal membrane oxygenation for respiratory or cardiac failure as well as hemodialysis) involve contact of the devices with blood which results in thrombus formation. Device thrombosis can result in thromboembolism, stroke, myocardial infarction, and device malfunction. Device thrombosis is a complex process that involves adhesion of blood molecules like fibrinogen and von Willebrand factor to the device surface followed by platelet adhesion, platelet activation, platelet aggregation, and clotting. In general, two approaches have been employed to solve the problem of device thrombosis: modification of the surface of the device to reduce thrombosis and administration of anti-clotting or anti-platelet drugs. However, even with all of the advances made, failure rates are still as high as 6%. Up to one-third of neonates and children suffer thrombosis complications from extracorporeal circulation devices. The most common approach is to add an anticoagulant like heparin systemically, but this can also lead to bleeding, thrombocytopenia, and hypertriglyceridemia (too much trigycerides). This disclosure provides methods and systems to prevent or reduce device thrombosis using nitrite and far-red light to reduce.

Nitric oxide is the endothelium-derived relaxing factor (EDRF) that modulates vascular tone by activating soluble guanylyl cyclase (sGC) in smooth muscle. In addition to acting as a vasodilator, NO reduces platelet activation, and reduces circulating blood cell adhesion to endothelia. The potential of NO to treat thrombosis is primarily through its action in reducing platelet activation, but as shown herein, it will also affect other aspects of clotting.

This disclosure describes methods and systems to combine nitrite and far-red light to treat thrombosis. In some aspects, this disclosure teaches that combined nitrite and far-red light treatment decreases coagulation related measures, whereas a single monotherapy is not effective—this supports a combination therapy. This disclosure precisely defines the effects of these therapies on a wider range of coagulation measures and provides mechanisms affecting efficacy so that the treatments can be optimized.

Many pathological conditions are partially caused by low NO bioavailability and/or would benefit from administration of NO including diabetes, atherosclerosis, peripheral artery disease, hypertension, hemolytic anemias, thrombotic disorders, wound healing, and heart failure with preserved ejection fraction. In many cases, NO administration would be most effective if it were targeted to areas of low oxygen (hypoxia) or ischemia.

This disclosure relates to methods as well as an innovative apparatus to treat device thrombosis.

Nitrite, rather than being biologically inert as suggested by some, is a physiologically and therapeutically relevant vasodilator, targeting NO release to areas of low oxygen, and involves a physiologically-relevant function of Hb and RBCs. Nitrite is unique among NO donating agents, being the only one that requires the presence of RBCs, whereas others' NO activity is blunted by RBCs. Nitrite bioactivation by RBCs involves nitrosyl-heme or nitrosothiol formation of a surface RBC thiol bond. Illumination with far-red light dramatically potentiates RBC-mediated nitrite bioactivation.

The present disclosure describes the effects of nitrite or the combination of nitrite and far-red light therapy for the prevention or reduction of device thrombosis, including the involvement of RBCs (FIG. 2).

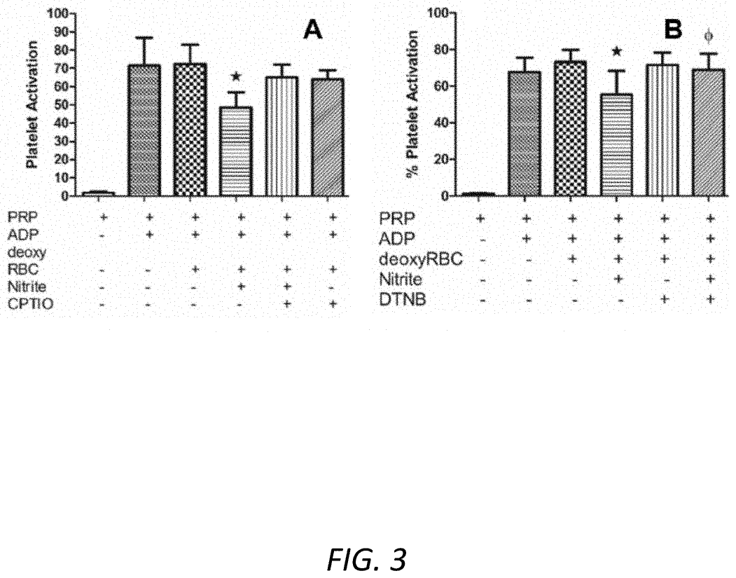

FIG. 2 shows that nitrite bioactivation as measured by inhibition of platelet activation, requires the presence of RBCs. Nitrite addition to activated platelets in the absence of RBCs has no effect (FIG. 2A—note scale is expanded). These data indicate that RBCs are responsible for nitrite bioactivation in blood. This interaction is depicted in FIG. 1. It has been shown that Hb is the primary erythrocytic nitrite reductase and that nitrite bioactivation by RBCs involves NO. FIG. 3A shows that nitrite-mediated inhibition of platelet activation by RBCs is abrogated when the NO scavenger CPTIO is present. Nitrite is the best suited NO donor for treating thrombosis as it requires the presence of RBCs to work while others efficacy is blunted by RBCs (via the reaction described in Equation 2). In addition, blocking RBC surface thiols using DTNB (FIG. 3B) abrogates inhibition of platelets.

The pathway through which NO scavenging is avoided is described herein. It is known that nitrite reacts with deoxygenated or partially deoxygenated hemoglobin to form NO and methemoglobin (Eq 1). The NO that comes off can bind another deoxygenated heme forming a ferrous nitrosyl species. This nitrosyl heme could come out of the hemoglobin and bind a surface thiol forming a nitrosyl heme thiolate. Thus, nitrosyl-heme exchange could be a mechanism for sGC activation. In this exchange, the two heme species that are most likely to be exported from hemoglobin are the ferric heme in methemoglobin and nitrosyl heme, exactly the two species made when nitrite is added to deoxyhemoglobin.

An intermediate in the nitrite hemoglobin reaction is a ferric nitrosyl species (methemoglobin with NO bound), and the NO can then come off and leave a ferric heme. The ferric heme nitrosyl can react with a thiol and form a nitrosothiol (which is a species that can elude NO scavenging and a candidate for our RBC membrane surface bound species). The importance of this pathway was tested by mixing hemoglobin, nitrite and thiols (e.g. glutathione which is present at 5 mM in the RBC) and measuring nitrosothiols using chemiluminescence assays. Experiments were performed at various oxygen tensions employing pre-mixed gases.

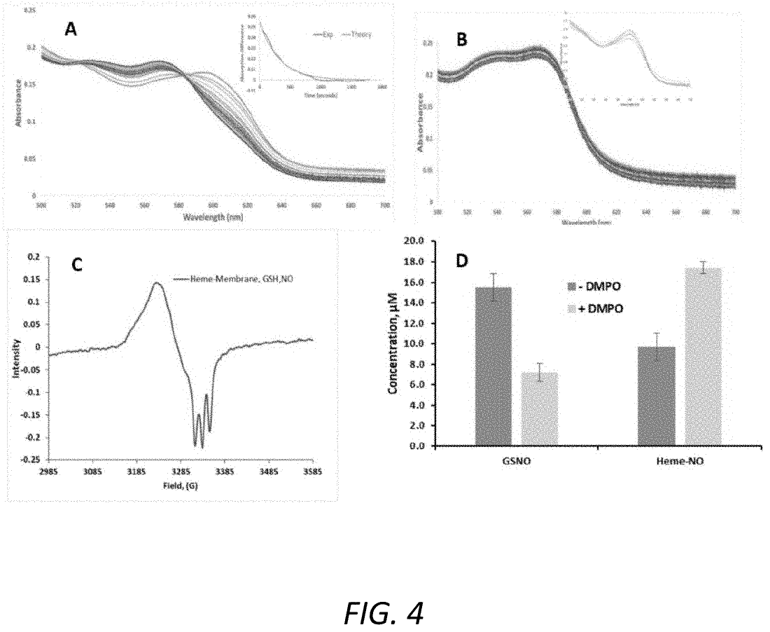

Ferric heme nitrosyl can itself be exported from the hemoglobin or the ferric heme can be exported and subsequently bind NO. Thus, one model system studied was hemin mixed with thiols and NO. As disclosed herein, thiols greatly accelerate formation of a ferrous heme nitrosyl and nitrosothiols from a mixture of thiol, hemin, and NO. These data are shown in FIG. 4. The heme was solubilized using either a 1:5 methanol:PBS mixture or added 10% RBC membranes with hemoglobin completely removed (confirmed by absorption spectroscopy). In the absence of glutathione (GSH), ferric hemin slowly forms a ferrous nitrosyl as expected by the classic reductive nitrosylation reaction with a half-life of about 10 minutes (FIG. 4A). However, in the presence of GSH (present in RBCs at about 5 mM) the reaction is over within the mixing time of the reagents (FIG. 4B). The formation of the ferrous nitrosyl species is confirmed using EPR spectroscopy (FIG. 4C). Importantly, the disclosed reactions generated a large amount of nitrosothiols (FIG. 4D) in accordance with the following reaction pathway

Fe^{III}+NO↔Fe^{III}NO

GSH+Fe^{III}NO→GS^{⋅}+Fe^{II}NO

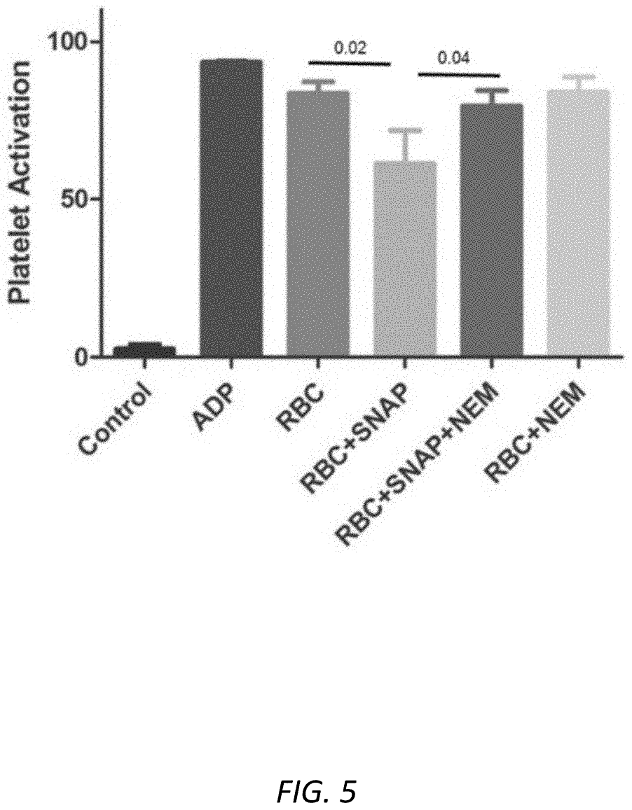

GS^{⋅}+NO→GSNO (Eq. 3)

where the ferric hemin reversibly binds NO, GSH reduces the hemin and forms a thiyl radical which then reacts with NO, similar to a mechanism proposed for DNIC formation. The formation of the thiyl radical is supported by lower GSNO yield when the thiyl radical trap 5,5-dimethyl-1-pyrroline N-oxide (DMPO) is added (FIG. 4D). Thus, this system of hemin, NO and thiol (all present in the RBC after nitrite addition) yield both species of interest: nitrosothiols and heme-nitrosyls (which can bind to a surface thiol). Studies using RBCs. FIG. 3B shows that when RBC surface thiols are blocked using DTNB, RBC mediated inhibition of platelet activation is abrogated; supporting a role for RBC surface thiols in nitrite bioactivation. Additionally, treating RBCs with the cell impermeable nitrosating agent S-Nitroso-N-acetylpenicillamine (SNAP) and then extensively washing the RBCs (removing excess SNAP) caused the RBCs to inhibit platelet activation. This inhibition was abrogated by pre-treatment with the thiol binding agent N-ethyl maleimide (NEM) (FIG. 5).

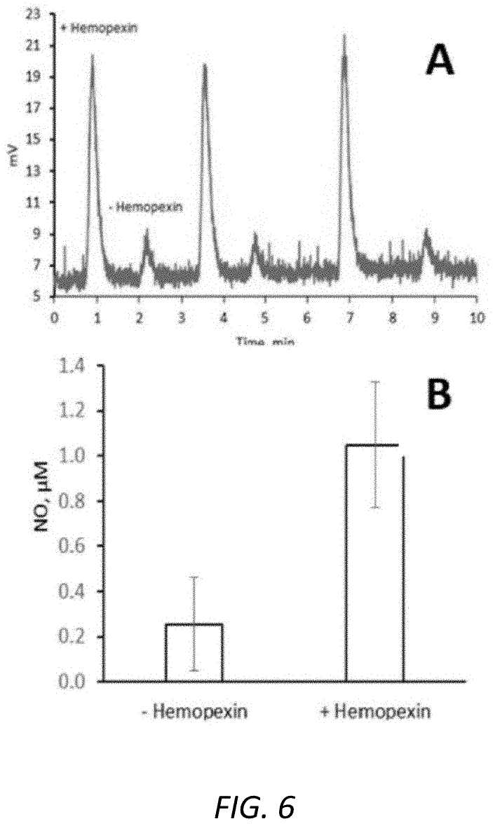

Nitrosyl hemes and nitrosothiols can be exported from the RBC following nitrite treatment. This was confirmed by treating RBCs with nitrite in platelet rich plasma (PRP) and measuring exported NO species after spinning down RBCs using chemiluminescence and EPR (for heme nitrosyls). In other experiments, the extent of heme nitrosyl export was determined using excess heme binding proteins such as albumin and hemopexin (a plasma protein with extremely high affinity for free heme that binds heme in plasma to prevent oxidative stress). FIG. 6 shows preliminary data examining heme nitrosyl export. To obtain the data disclosed in FIG. 6, deoxygenated RBCs were incubated with nitrite in the presence or absence of hemopexin, spun down the RBCs, and measured NO species in the supernatant using a chemiluminescence assay (the tri-iodide assay). Although some NO species were expected due to hemolysis, significantly greater NO was observed in samples that contained hemopexin compared to those that did not contain hemopexin (FIG. 6) despite the fact that samples with hemopexin had less hemolysis than samples without hemopexin (35±7 vs 93±20 μM Hb, n=4, data not shown). These data support nitrosyl-heme export consistent with hemopexin capture of nitrosyl heme.

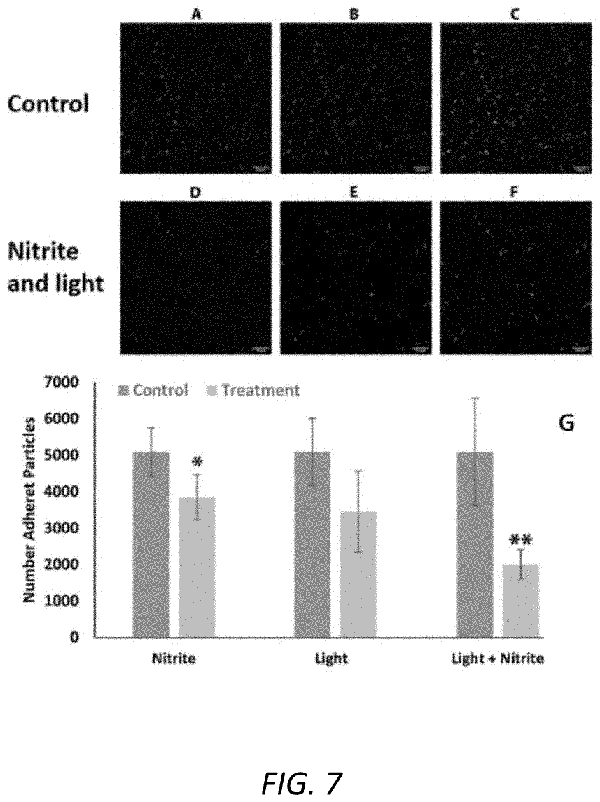

Also disclosed herein is a mechanism though which nitrate potentiates the anti-thrombotic effects of far-red light. FIG. 2 demonstrates the dramatic effect of combining far-red light on inhibiting platelet activation by RBCs. As disclosed above, and illustrated in FIG. 1, this effect is due to photolysis of a NO species bound to a RBC surface thiol. Importantly, it has also been shown that the effect of the light is not due to heating by comparing effects at different temperatures. The effects of combining far-red light and nitrite in inhibiting several different measures of clotting using different agonists are disclosed herein. Particularly relevant to device thrombosis, as shown in FIG. 7, dual treatment decreases platelet adhesion in a microchannel assay.

Although the combination of nitrite and light is universally effective in reducing clotting in all assays conducted, monotherapies with either nitrite or light had mixed effects. This is due to not enough NO being generated in the monotherapies in some conditions.

Studies Using Model Compounds.

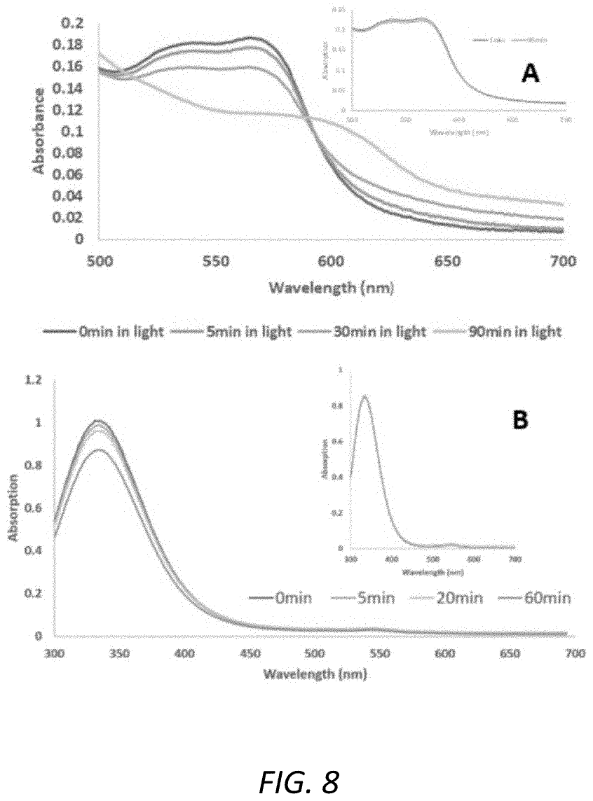

The direct effects of 660 nm light on model compounds were studied to test the effects of light in accordance with FIG. 1. The photolysis rate depends on absorption cross section, quantum yield and actinic flux, all of which are wavelength dependent. The direct effects of far-red light exposure were tested by exposing various nitrosothiols (since absorbance will be slightly different for different ones) such as GSNO, CSNO and SNO-albumin and heme nitrosyls in the presence of thiols. The effects of light wavelength (using different diodes), intensity and duration of illumination were also tested. FIG. 8A shows preliminary data on the effect of far-red light on the heme-nitrosyl mixture in the presence of thiols and FIG. 8B shows similar data for GSNO. These data support the paradigm shown in FIG. 1. These results were verified using additional analytical techniques including chemiluminescence and EPR to verify species. Further, experiments were conducted using a more physiologically relevant system; using RBC membranes to solubilize the heme (for *A) instead of methanol.

In order to further test the role of heme-nitrosyls and/or nitrosothiols in far-red light potentiated inhibition of platelet activation, these compounds were added to PRP and platelet activation was measured along with the effects of light. Wavelength and oxygen tension dependence was elucidated.

Studies Using RBCs.

In FIG. 5, SNAP was added and our heme-nitrosyl mixtures to RBC excess compounds were washed out, and the effects on platelet activation by light illumination were examined. Light potentiates effects using SNAP or heme-nitrosyls or both. In FIG. 6, the effects of light on heme nitrosyl export (like in FIG. 6) and nitrosothiol export were tested.

Studies in Blood.

The effects of nitrite and light on reducing clotting in blood were conducted by comparing sham, light alone, nitrite alone and the combination. Platelet activation was measured using flow cytometry assays employing various agonists When RBCs are present, nitrite affects several parameters measured using TEG including increasing the time to initial fibrin formation (R-time), decreasing the rate of clot growth.

Data disclosed herein strongly supports the role of NO in RBC-mediated inhibition of platelet activation by nitrite using NO scavengers that abrogate the inhibitory effects (e.g. FIG. 3A).

Optimal conditions involving nitrite concentrations, oxygen tensions, wavelengths, and illumination durations were tested in the platelet adhesion microchannel assay (FIG. 7) and compared to effects already observed using 660 nm. These experiments were important for the design of the methods, devices, and systems disclosed herein as platelet adhesion plays a major role in device thrombosis.

Device thrombosis is a significant complication in otherwise life-saving interventions including hemodialysis catheters, stents, extracorporeal devices (including those used in cardiopulmonary bypass surgery and extracorporeal membrane oxygenation (ECMO), and hemodialysis), and in left and right ventricular assist devices. Clotting is a major daily problem in continuous renal replacement therapy (CRRT). Standard care for preventing device thrombosis involves use of systemic anti-coagulant but this can lead to bleeding and, in some cases, heparin-induced thrombocytopenia. NO has been employed as a potential alternative preventative agent, but these have suffered from distribution of NO due to its short half-life and challenges in the kinetics of NO-releasing agents. This disclosure provides for the use of nitrite as an NO donor to reduce device thrombosis and nitrite is in fact the most appropriate agent to use as unlike other NO donors that are less effective in the presence of RBCs, nitrite requires the presence of RBCs for efficacy (see FIG. 2). Far and infrared light has also been shown to have positive effects in extracorporeal circulation circuits. The presently disclosed methods and systems combine nitrite and light treatment with in-line administration that can easily be incorporated in extracorporeal circulation circuits. These methods and systems improve over previous attempts in that (1) NO is an ideal agent to prevent device thrombosis due to its anti-platelet activity, (2) nitrite is the best NO-based anti-thrombotic agent due to the fact that its activity is dependent on the presence of red blood cells whereas other NO agents' activity is blunted by red blood cells, (3) nitrite is found naturally in the body and has been studied as a therapeutic in other applications, and (4) the light provides localized treatment so that systemic effects are avoided and the duration and frequency of treatment is easily controlled. A system for preventing device thrombosis is disclosed. Some design parameters employed include nitrite dose, wavelength, illumination duration, and light intensity. Light sources (LEDs) using 660 nm light was tested. Other light sources could also be used that are more readily available or which achieve greater tissue penetration. Preliminary data shown below using this wavelength.

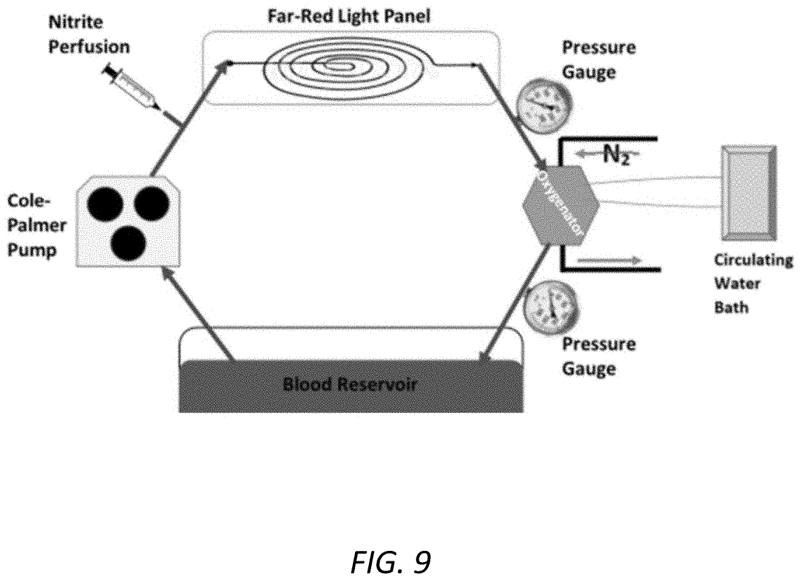

In some embodiments of the presently disclosed methods and systems, nitrite is continuously infused in line with the extracorporeal circuit and far-red light (or other wavelengths based in results from previous aims) illumination is also administered in-line and the duration of illumination is dictated by the volume of tubing in the illuminated area and pump speed. In other embodiments, nitrite is discontinuously infused in line with the extracorporeal circuit. The tubing in the illumination volume is controlled by winding it as illustrated in FIG. 9 that shows a schematic of the prototype testing device. Two Mito MD LED sources were employed with the tubing sandwiched in between. The power of the LEDs is reported as 100 mW/cm² at 6 inches from the device and we measured 35 mW/cm² at this distance and 230 mW/cm² directly at the surface. Lower powers can be achieved with filters. These lamps contain fans so that heating is not an issue. The system was also maintained at the desired temperature using a circulating water bath attached to the oxygenator representative of use conditions. The blood was deoxygenated by flowing nitrogen through the oxygenator to mimic venous blood from a real patient and hemoglobin saturation was about 70% or so, as tested using absorption spectroscopy and our integrating sphere detector for turbid samples.

Different extracorporeal devices require different flow rates. In the microchannel assay (FIG. 7) the sample was illuminated for 48 seconds. For continuous renal replacement therapy (CRRT), flow rates are about 150 mL/min. Only 120 mL of blood in CRRT would need to be illuminated to get the duration as in our microchannel device This disclosure provides the minimum effective duration.

The present disclosure provides methods and systems for inhibiting device thrombosis for a patient. In one aspect, a method for inhibiting device thrombosis for a patient is provided. In one aspect, the method may include circulating blood through an extracorporeal circuit coupled to the patient, infusing nitrite into the blood such that the nitrite is bioactivated by red blood cells to form nitric oxide, and illuminating the blood with far-red light to increase bioactivation of the nitrite by the red blood cells. In some aspects, circulating blood means passing blood through a section of tubing of the circuit. In some aspects, nitrite can include a pharmaceutically acceptable nitrite salt. In some aspects, infusing nitrite into the blood can include adding nitrite to the blood as it circulates through a section of tubing of the circuit. In some examples, nitrite infusion is accomplished by delivering nitrite in a pharmaceutically stable salt into a section of tubing of the circuit that contains blood. Nitrite infusion (rate and amount) can be controlled either manually, for example, by a plunger, or automatically, for example, by an electronic controller.

As used herein, “blood” refers to whole blood, red blood cells, leukoreduced blood, plasma, and serum.

In some aspects, infusing the nitrite into the blood may include infusing the nitrite in line with the extracorporeal circuit. In some aspects, infusing the nitrite into the blood may include continuously infusing the nitrite for a period of time while the blood circulates through the extracorporeal circuit. In some aspects, illuminating the blood with the far-red light may include illuminating the blood with the far-red light in line with the extracorporeal circuit. In some aspects, illuminating the blood in line with the extracorporeal circuit means illuminating the blood with far-red light while the blood circulates through the extracorporeal circuit. In some examples, illuminating the blood in line with the extracorporeal circuit means illuminating the blood with far-red light contemporaneous with infusion of nitrite into the blood.

In some aspects, illuminating the blood with the far-red light may include continuously illuminating the blood with the far-red light for a period of time while the blood circulates through the extracorporeal circuit. In some aspects, illuminating the blood with the far-red light may include illuminating the blood for a period of time at a plurality of discontinuous segments along one or more portions of the extracorporeal circuit.

In some aspects, illuminating the blood with the far-red light may include passing the blood through a section of tubing disposed between a pair of light sources. In some aspects, the section of tubing may be arranged in a coil. In some aspects, the pair of light sources may include a pair of light-emitting diodes. In some aspects, illuminating the blood with far-red light can include illuminating the blood with the far-red light emitted at wavelengths of about 600 nm to about 800 nm. For example, illuminating the blood with far-red light can include illuminating the blood with the far-red light emitted at wavelengths of about 600 nm to about 750 nm. In some examples, illuminating the blood with far-red light can include illuminating the blood with the far-red light emitted at wavelengths of about 600 nm to about 700 nm. In some examples, illuminating the blood with far-red light can include illuminating the blood with the far-red light emitted at wavelengths of about 610 nm to about 690 nm. In some examples, illuminating the blood with far-red light can include illuminating the blood with the far-red light emitted at wavelengths of about 620 nm to about 680 nm. In some examples, illuminating the blood with far-red light can include illuminating the blood with the far-red light emitted at wavelengths of about 630 nm to about 680 nm. In some examples, illuminating the blood with far-red light can include illuminating the blood with the far-red light emitted at wavelengths of about 640 nm to about 680 nm. In some examples, illuminating the blood with far-red light can include illuminating the blood with the far-red light emitted at wavelengths of about 650 nm to about 670 nm. In some examples, illuminating the blood with far-red light can include illuminating the blood with the far-red light emitted at a wavelength of about 660 nm. In some aspects, illuminating the blood with the far-red light can include illuminating the blood with the far-red light emitted at a wavelength of 660 nm. In some aspects, the method also may include maintaining the blood circulating through the extracorporeal circuit at a predetermined temperature using a circulating water bath.

Also disclosed is a system for inhibiting device thrombosis for a patient is provided. In one aspect, the system may include an extracorporeal circuit configured to circulate a blood of the patient, a nitrite infusion device coupled to the extracorporeal circuit and configured to infuse nitrite into the blood such that the nitrite is bioactivated by red blood cells to form nitric oxide, and one or more light sources disposed along the extracorporeal circuit and configured to illuminate the blood with far-red light to increase bioactivation of the nitrite by the red blood cells.

In some aspects, the nitrite infusion device may be configured to infuse the nitrite in line with the extracorporeal circuit. In some aspects, the nitrite infusion device may be configured to infuse the nitrite for a period of time while the blood circulates through the extracorporeal circuit. In some aspects, the one or more light sources may be configured to illuminate the blood with the far-red light in line with the extracorporeal circuit. In some aspects, the one or more light sources may be configured to illuminate the blood with the far-red light for a period of time while the blood circulates through the extracorporeal circuit. In some aspects, the extracorporeal circuit may include a section of tubing disposed adjacent to the one or more light sources. In some aspects, the section of tubing may be arranged in a coil. In some aspects, the one or more light sources may include a pair of light sources, and the section of tubing may be disposed between the pair of light sources. In some aspects, the pair of light sources may include a pair of light-emitting diodes. In some aspects, the one or more light sources may be configured to emit the far-red light at a wavelength of 660 nm. In some aspects, the system also may include a circulating water bath configured to maintain the blood circulating through the extracorporeal circuit at a predetermined temperature.

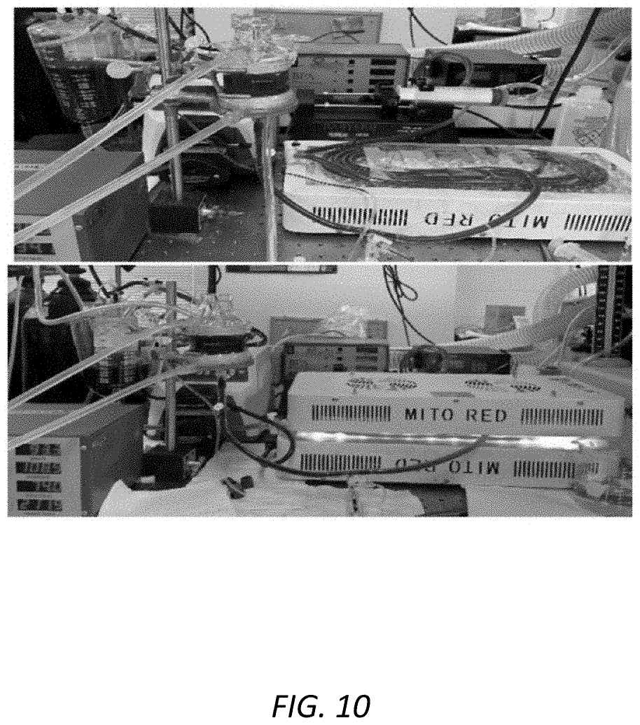

A prototype device is pictured in FIG. 10. Disclosed herein are outcome measures to test the efficacy of the device, including:
(1) Patency of the device. Whether enough clotting will occur to impede flow enough so that the experiment needs to be stopped before six hours of testing. The time to stopping was recorded.
(2) Pressure as a function of time. The pressure in the circuit was recorded every 15 minutes with higher pressures indicating occlusive clotting.
(3) Visual inspection for clotting. Tubing was examined thoroughly every 15 minutes with flashlights and photographs were taken to record the size and number of visual clots.
(4) Blood aliquots were collected every 15 minutes to measure oxygenation state of hemoglobin (so the nitrogen flow can be adjusted). A spectrometer was used with an integrating sphere that collected scattered light to get true absorption of turbid samples. Fitting spectra to basis spectra gives the percent oxygenation.
(5) Platelet activation was measured using fluorescent antibodies and fixing the samples for flow cytometry.
(6) Hemolysis. RBCS were sedimented and hemoglobin concentration in the supernatant determined by absorption spectroscopy.
(7) Platelet count was assessed as part of a complete blood count. Platelet count goes down as platelets adhere and aggregate in the system.

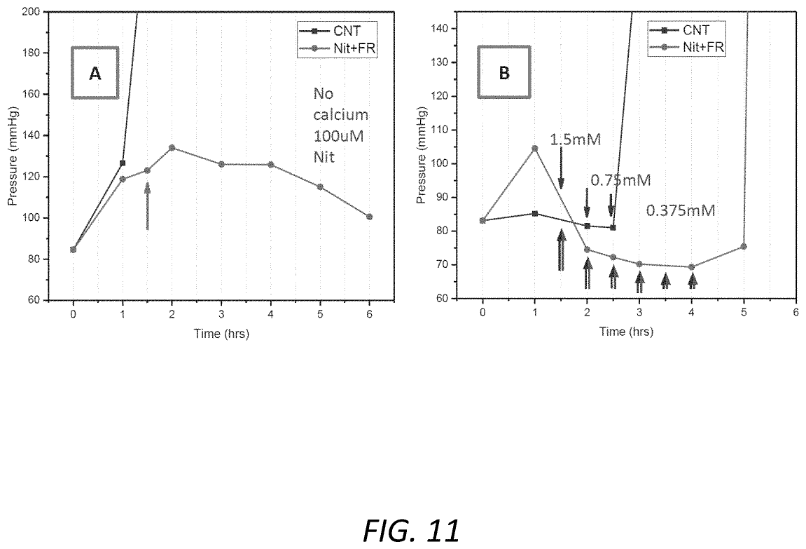

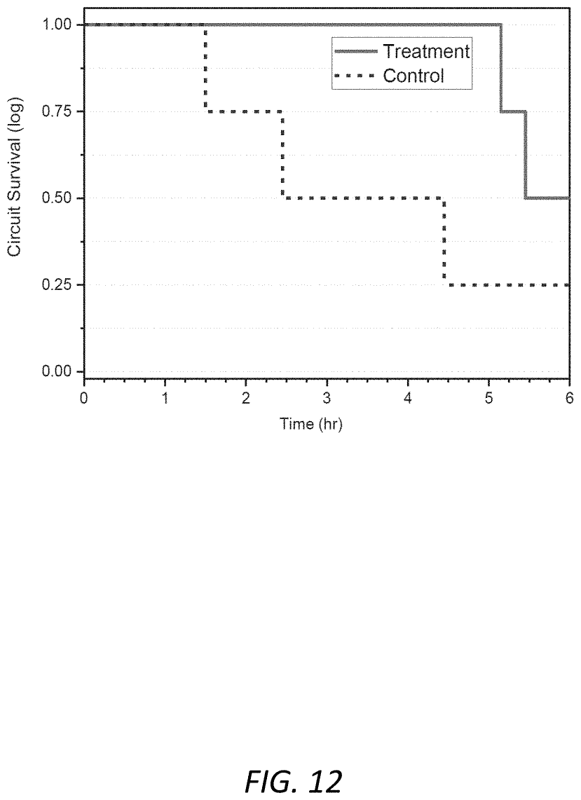

In addition, nitrite was measured to assess the effective dose that a patient may encounter. The reaction of the nitrite with hemoglobin was pseudo first order in hemoglobin which is present at 10 mM (in heme) in blood. With a bimolecular rate constant of 1 M⁻¹s⁻¹ for partially deoxygenated hemoglobin, the lifetime of the nitrite was approximated at about 100 seconds so most was consumed in the circuit. FIG. 11 shows data collected using the device. To collect this data, whole blood was circulated for up to 6 hours. These data were collected on whole blood that was refrigerated, which reduces platelet function. Thus, for some cases where pressure did not increase in the system, calcium was infused up to 1.15 mM total so as to enhance clotting. For all experiments, the same blood was used for control and treatment and the same amount of calcium infused at the same timepoints. For the example shown in FIG. 11A, no calcium was infused. Nitrite (100 μM final concentration) and light treatment was started at the 1.5 hour mark. It was seen that the pressure in the control went up rapidly at the 1.5 hour mark and this was just before the system lost patency—the tubing actually became disconnected. For the example shown in FIG. 11B, calcium additions was added at the 1.5 hour mark and at each of the next two 0.5 hour intervals (1.5 mM; 0.75 mM; and 0.375 mM). Nitrite and far-red light therapy were mixed with calcium at 0.5 hour intervals beginning the 1.5 hour mark and continuing until the 4 hour mark (Ca+50 uM Nit; Ca+25 uM Nit; Ca+12.5 uM Nit; 0.375 mM Ca+12.5 uM Nit; 0.25 mM Ca+8.3 uM Nit; 0.25 mM Ca+8.3 uM Nit). It was seen that pressure in the treatment circuit decreased to less than that of the control circuit once nitrite treatment combined with far-red light therapy was added. FIG. 12 shows a Kaplan Meier graph of circuit survival over time for four separate experiments, indicating that circuit survival improved significantly with treatment. Overall, these experiments showed that the combined light/nitrite treatment had potential to prevent device thrombosis.

Also disclosed herein is a system consisting of two circuits so as to compare control to treatment (or compare two different treatments) run at the exact same time. The system was tested by comparing treatment to control in terms of patency, platelet count, and platelet activation. Both patency and platelet count show that the treatment is beneficial. More specifically, two identical circuits were set up with equal lengths of tubing, the same number of connection points, and similar connecting valves and injection sites. One circuit served as the “control circuit” and the other served as the “treated circuit”. Both systems were connected to a Harvard Apparatus pump (PHD 2000) for intermittent infusions, the same nitrogen tank, and a Haake temperature control circulating water bath, and both were run simultaneously. An amount of normal saline (250 mL) was degassed in both circuits under 37° C. temperature for an hour. Next, the RBC+PRP mixture containing 230 mL packed RBCS, 230 mL saline, and 60 mL PRP was divided into two 250 mL portions and divide between reservoirs of both treated and control systems. The pump was then started and blood circulation initiated such that nitrite/saline infusion began. Immediately thereafter, both Mitored light panels were illuminated in the treatment loop and remained on until the end of the experiment. Depending on the variations in pressure over time, gradual addition of calcium chloride became required to initiate clotting in the systems. Survival of the circuits was considered failed when the pressure before the oxygenator reached 260 mmHg which correlates with very low blood pressure after the oxygenator (less than 10 mmHg).

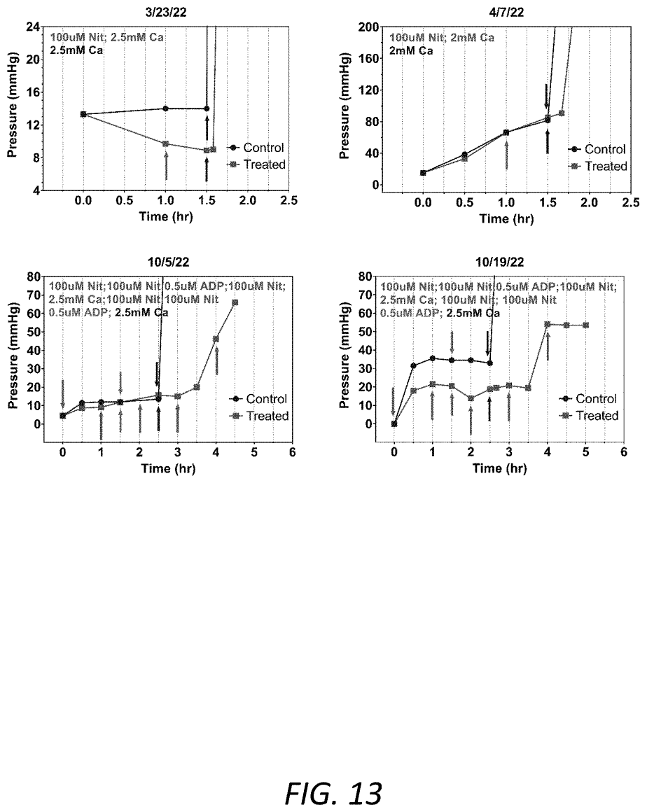

FIG. 13 shows a trace of pressure change in both the control (in blue) and the treated (in red) systems on four different days of simultaneously running both circuits. However, the range of the pressure varied among these experiments, platelet activation in both systems was boosted by ADP (light blue arrows) and/or calcium (dark blue arrows) until the control system clotted. The treated system had the advantage of 100 uM nitrite hourly infusion (red arrows) while the light panels illuminated the circulating blood. The same volume of saline (pH 7.4) was the infusion substance added to the control system at the same time as nitrite dosing (RBC:saline:PRP was 3.8:3.8:1 in the top two panels of FIG. 13, and 1.7:1.7:1 for the two bottom panels of FIG. 13). Each of the infusions were performed using a Harvard pump at the same rate of the pumping blood (150 mL/min) and mean blood pressure in both systems was tracked every 30 minutes. In all experiments, the treated circuit survived longer under the same addition of platelet activators as in the control circuits. Thus, FIG. 13 represents the increased circuit survival time in the treated systems.

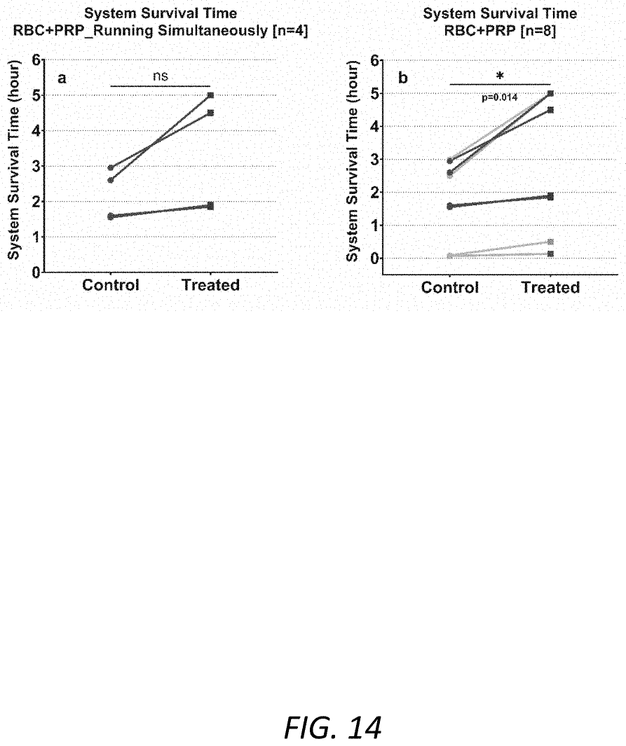

FIG. 14 shows line graphs of survival proportions between control and treatment circuits. FIG. 14a shows the line graph of the four experiments illustrated in FIG. 13, when the circuits were running simultaneously, with the longer survival time being in the circuit treated with nitrite and light illumination. FIG. 14b shows the line graph of the results of eight experiments all with a mixture of red blood cells and platelet-rich plasma. Green lines represent simultaneous runs, orange lines represent 2-day experiments, and purple lines represent dame day experiments performed in sequence.

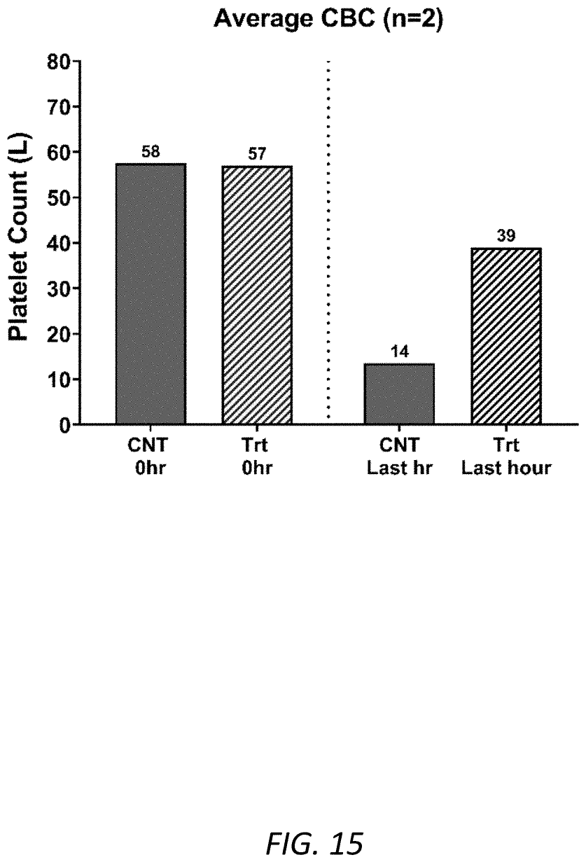

In addition to circuit survival, FIG. 15 shows noticeable effects of the treatment on the platelet counts. Averaged data collected from Complete Blood Count showed a 32% decrease in the number of platelets in the treated system from the first to the last sample collected from the circuits. This value for the control system was a 76% loss.

The mentioned protocol is the most updated and optimized method for the device thrombosis prototype. Prior to the four trials above, four more experiments were completed with the mixture of packed red blood cells with platelet-rich plasma, as shown in FIG. 14b. In two experiments (shown in orange) day one was dedicated to the control system, and day two to the treated system following the same—if any—calcium addition time, volume, and concentrations. The treated circuit is shown in purple in FIG. 14b. FIG. 14b demonstrates the outcome of all eight experiments with RBC+PRP mixtures. Altogether, the synergistic effects of nitrite infusion and red light treatment are significant when compared to untreated blood (paired t-test value *p=0.014). This result is in line with previously published findings in microscale experiments.

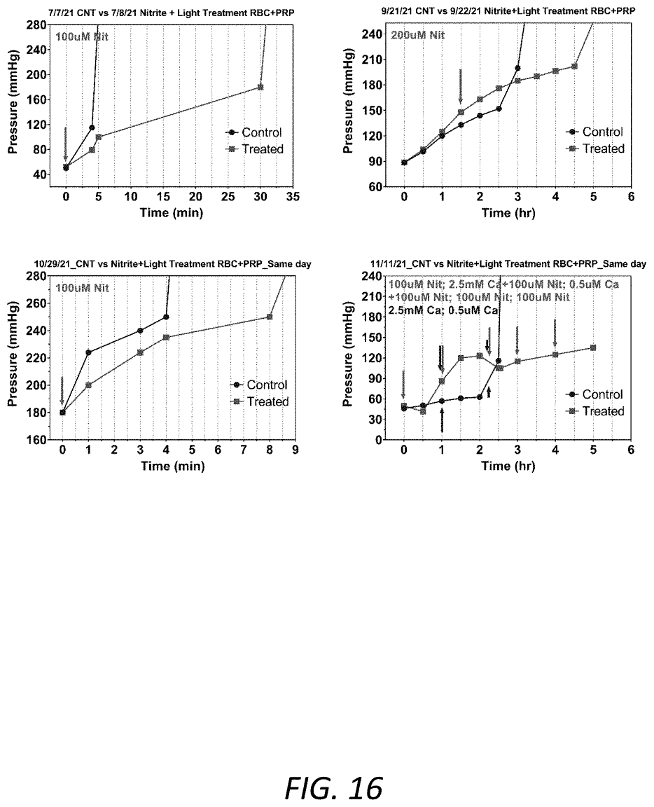

FIG. 16 shows the effects of the treatment on the system survival time in the 2-day and same-day trials. The top two panels were collected in 2-day experiments while the bottom two were from the same-day runs. Regardless of the survival time length, treated system always showed prolonged survival. Red arrows represent hourly 100 uM nitrite infusions and dark blue arrows represent calcium infusions. In these experiments, RBC:saline:PRP was 3.8:3.8:1.

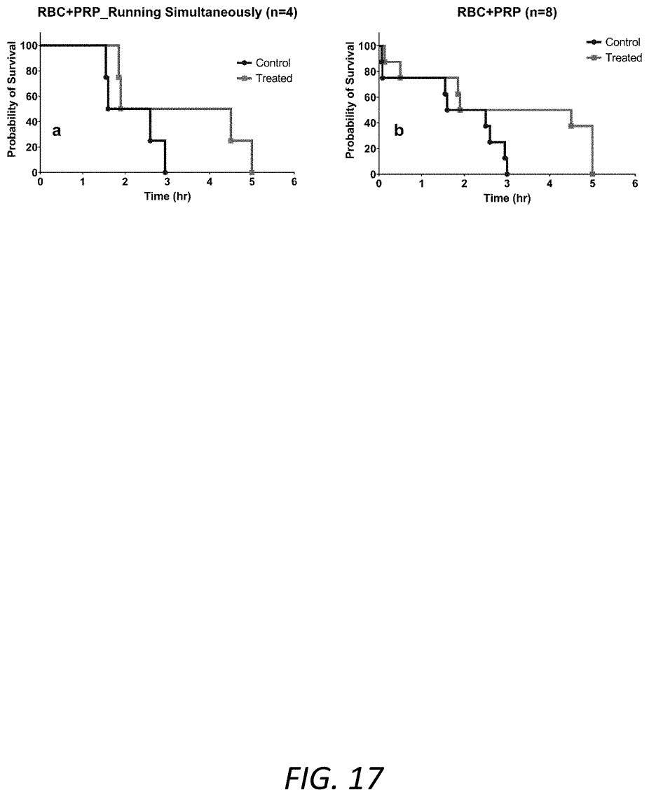

FIG. 17 shows the Kaplan Meier survival curves of the simultaneous (panel a) and eight combined runs with red blood cells mixed with platelets (panel b).

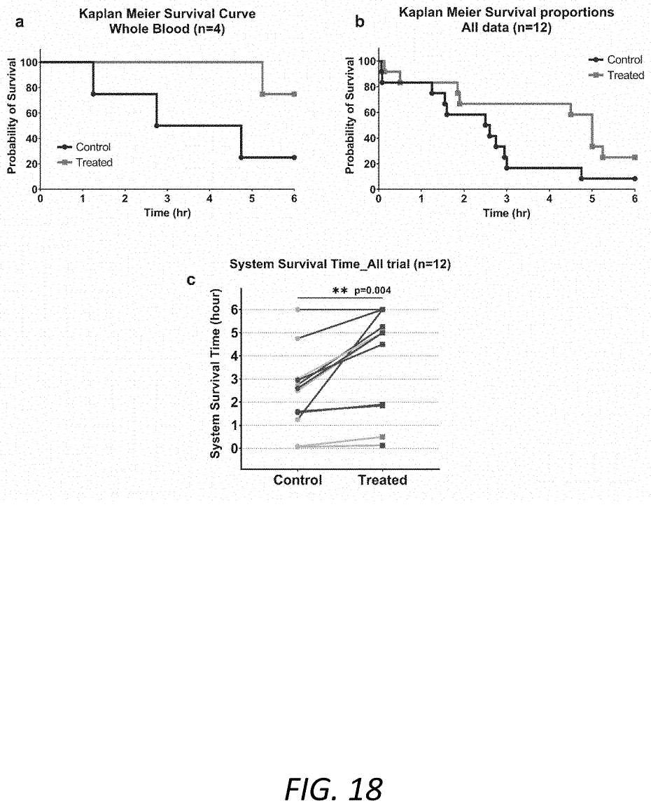

FIG. 18 shows Kaplan Meier survival curves and a line graph of system survival. The Kaplan Meier survival time curves in FIG. 18a are based on data obtained from four experiments with whole blood. The Kaplan Meier survival time curves in FIG. 18b are based on data obtained from the four trials of FIG. 18a combined with the other eight experiments involving RBC+PRP. FIG. 18c is a line graph of all twelve successful paired runs.

Early testing involved the use of whole blood bags delivered on ice which led to initially activated platelets and consequently high mean blood pressure at the start point. However, the modified method (mixing RBC: saline: PRP) showed significant results. FIG. 18 represents the collected data from tests using whole blood. Once all twelve successful paired runs are put together the significance is evident (FIG. 18c, **p=0.004).

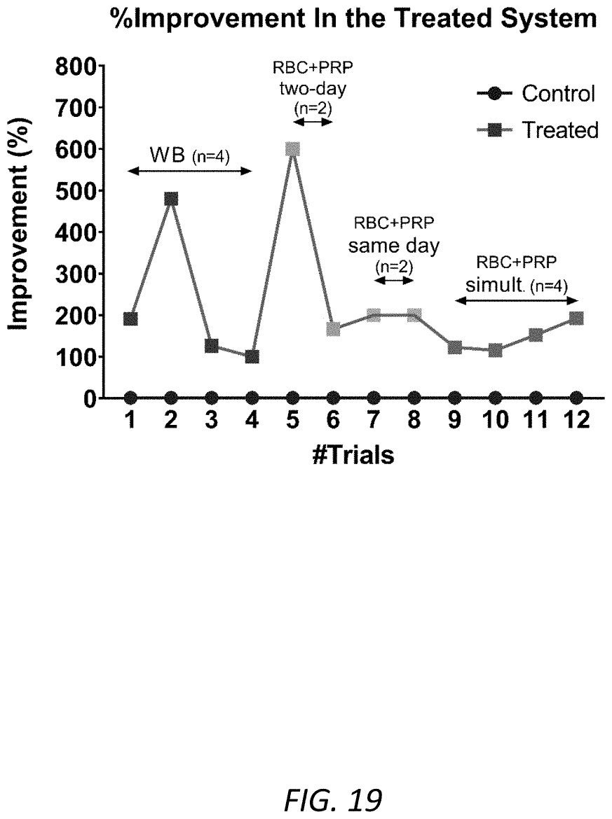

FIG. 19 shows the percentage range of the improvement of the combined treatment of nitrite and red light in improving the survival time in the treated systems in a range of 100% to 600%. On average, the survival time of the treated system is 220% longer than the control circuit.

Although specific embodiments of the disclosure have been described, one of ordinary skill in the art will recognize that numerous other modifications and alternative embodiments are within the scope of the disclosure. For example, while various illustrative implementations and structures have been described in accordance with embodiments of the disclosure, one of ordinary skill in the art will appreciate that numerous other modifications to the illustrative implementations and structures described herein are also within the scope of this disclosure. Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments.
1 Wajih, N., Liu, X., Shetty, P., Basu, S., Wu, H., Hogg, N., Patel, R. P., Furdui, C. M. & Kim-Shapiro, D. B. The role of red blood cell S-nitrosation in nitrite bioactivation and its modulation by leucine and glucose. Redox Biol 8, 415-421, doi:10.1016/j.redox.2016.04.004 (2016). PMC4864376
2 Liu, C., Wajih, N., Liu, X., Basu, S., Janes, J., Marvel, M., Keggi, C., Helms, C. C., Lee, A. N., Belanger, A. M., Diz, D. I., Laurienti, P. J., Caudell, D. L., Wang, J., Gladwin, M. T. & Kim-Shapiro, D. B. Mechanisms of human erythrocytic bioactivation of nitrite. The Journal of biological chemistry 290, 1281-1294, doi:10.1074/jbc.M114.609222 (2015). PMC4294492
3 Sukavaneshvar, S. Device thrombosis and pre-clinical blood flow models for assessing antithrombogenic efficacy of drug-device combinations. Advanced Drug Delivery Reviews 112, 24-34, doi:10.1016/j.addr.2016.07.009 (2017).
4 Wajih, N., Basu, S., Ucer, K. B., Rigal, F., Shakya, A., Rahbar, E., Vachharajani, V., Guthold, M., Gladwin, M. T., Smith, L. M. & Kim-Shapiro, D. B. Erythrocytic bioactivation of nitrite and its potentiation by far-red light. Redox Biology 20, 442-450, doi:10.1016/j.redox.2018.11.001 (2019). PMC6230921
5 Selleng, S. & Selleng, K. Heparin-induced thrombocytopenia in cardiac surgery and critically ill patients. Thromb. Haemost. 116, 843-851, doi:10.1160/th16-03-0230 (2016).
6 Shore-Lesserson, L., Baker, R. A., Ferraris, V. A., Greilich, P. E., Fitzgerald, D., Roman, P. & Hammon, J. W. The Society of Thoracic Surgeons, The Society of Cardiovascular Anesthesiologists, and The American Society of ExtraCorporeal Technology: Clinical Practice Guidelines*-Anticoagulation During Cardiopulmonary Bypass. Annals of Thoracic Surgery 105, 650-662, doi:10.1016/j.athoracsur.2017.09.061 (2018).
7 Stamler, J. S. & Loscalzo, J. The Antiplatelet Effects of Organic Nitrates and Related Nitroso-Compounds Invitro and Invivo and Their Relevance to Cardiovascular Disorders. J Am Coll Cardiol 18, 1529-1536 (1991).
8 Loscalzo, J. Nitric oxide insufficiency, platelet activation, and arterial thrombosis. Circ. Res. 88, 756-762 (2001).
9 Voetsch, B., Jin, R. C. & Loscalzo, J. Nitric oxide insufficiency and atherothrombosis. Histochem. Cell Biol. 122, 353-367, doi:10.1007/s00418-004-0675-z (2004).
10 Radomski, M. W., Palmer, R. M. J. & Moncada, S. THE ROLE OF NITRIC-OXIDE AND CGMP IN PLATELET-ADHESION TO VASCULAR ENDOTHELIUM. Biochem. Biophys. Res. Commun. 148, 1482-1489, doi:10.1016/s0006-291x(87)80299-1 (1987).
11 Vilahur, G., Baldellou, M. I., Segales, E., Salas, E. & Badimon, L. Inhibition of thrombosis by a novel platelet selective S-nitrosothiol compound without hemodynamic side effects. Cardiovasc. Res. 61, 806-816, doi:10.1016/j.cardiores.2003.11.034 (2004).
12 Vilahur, G. & Badimon, L. in New Therapeutic Agents in Thrombosis and Thrombolysis (eds Jane E. Freedman & Joseph Loscalzo) Ch. 29, 499-516 (Informa Healthcare, 2009).
13 Karmohapatra, S. K., Chakraborty, K., Kahn, N. N. & Sinha, A. K. The role of nitric oxide in aspirin induced thrombolysis in vitro and the purification of aspirin activated nitric oxide synthase from human blood platelets. Am J Hematol 82, 986-995, doi:10.1002/ajh.20955 (2007).
14 Jana, P., Maiti, S., Kahn, N. N. & Sinha, A. K. Estriol-induced fibrinolysis due to the activation of plasminogen to plasmin by nitric oxide synthesis in platelets. Blood Coagul. Fibrinolysis 26, 316-323, doi:10.1097/mbc.0000000000000085 (2015).
15 Teligui, L., Dalmayrac, E., Mabilleau, G., Macchi, L., Godon, A., Corbeau, J. J., Denomme, A. S., Bouquet, E., Boer, C. & Baufreton, C. An ex vivo evaluation of blood coagulation and thromboresistance of two extracorporeal circuit coatings with reduced and full heparin dose. Interactive Cardiovascular and Thoracic Surgery 18, 763-769, doi:10.1093/icvts/ivu011 (2014).
16 Lauer, T., Preik, M., Rassaf, T., Strauer, B. E., Deussen, A., Feelisch, M. & Kelm, M. Plasma nitrite rather than nitrate reflects regional endothelial nitric oxide synthase activity but lacks intrinsic vasodilator action. Proc. Natl. Acad. Sci. USA 98, 12814-12819 (2001).
17 Cosby, K., Partovi, K. S., Crawford, J. H., Patel, R. P., Reiter, C. D., Martyr, S., Yang, B. K., Waclawiw, M. A., Zalos, G., Xu, X. L., Huang, K. T., Shields, H., Kim-Shapiro, D. B., Schechter, A. N., Cannon, R. O. & Gladwin, M. T. Nitrite reduction to nitric oxide by deoxyhemoglobin vasodilates the human circulation. Nat. Med. 9, 1498-1505 (2003).
18 Gladwin, M. T., Schechter, A. N., Kim-Shapiro, D. B., Patel, R. P., Hogg, N., Shiva, S., Richard O Cannon, I., Kelm, M., Wink, D. A., Espey, M. G., Oldfield, E. H., Pluta, R. M., Freeman, B. A., Jack R Lancaster, J., Feelisch, M. & Lundberg, J. O. The emerging biology of the nitrite anion. Nature Chemical Biology 1, 308-314 (2005).
19 Huang, Z., Shiva, S., Kim-Shapiro, D. B., Patel, R. P., Ringwood, L. A., Irby, C. E., Huang, K. T., Ho, C., Hogg, N., Schechter, A. N. & Gladwin, M. T. Enzymatic function of hemoglobin as a nitrite reductase that produces Nitric oxide under allosteric control. J. Clin. Invest. 115, 2099-2107 (2005).
20 Kim-Shapiro, D. B. The reaction between nitrite and deoxyhemoglobin. Reassessment of reaction kinetics and stoichiometry. Comparative biochemistry and physiology. Part A, Molecular & integrative physiology 280, 31126-31131, doi:10.1016/j.cbpb.2005.04.016 (2005).
21 Lundberg, J. O. et al. Nitrate and nitrite in biology, nutrition and therapeutics. Nature Chemical Biology 5, 865-869, doi:10.1038/nchembio.260 (2009). Pmc3226741
22 Wajih, N., Basu, S., Jailwala, A., Kim, H. W., Ostrowski, D., Perlegas, A., Bolden, C. A., Buechler, N. L., Gladwin, M. T., Caudell, D. L., Rahbar, E., Alexander-Miller, M. A., Vachharajani, V. & Kim-Shapiro, D. B. Potential therapeutic action of nitrite in sickle cell disease. Redox Biol 12, 1026-1039, doi:10.1016/j.redox.2017.05.006 (2017). PMC5430577
23 Wajih, N., Alipour, E., Rigal, F., Zhu, J., Perlegas, A., Caudell, D. L. & Kim-Shapiro, D. Effects of nitrite and far-red light on coagulation. Nitric Oxide 107, 11-18 (2021). PMC7855911
24 Srihirun, S., Sriwantana, T., Unchern, S., Kittikool, D., Noulsri, E., Pattanapanyasat, K., Fucharoen, S., Piknova, B., Schechter, A. N. & Sibmooh, N. Platelet Inhibition by Nitrite Is Dependent on Erythrocytes and Deoxygenation. PLOS One 7, e30380 (2012).
25 Cosby, K., Partovi, K. S., Crawford, J. H., Patel, R. P., Reiter, C. D., Martyr, S., Yang, B. K., Waclawiw, M. A., Zalos, G., Xu, X., Huang, K. T., Shields, H., Kim-Shapiro, D. B., Schechter, A. N., Cannon, R. O., 3rd & Gladwin, M. T. Nitrite reduction to nitric oxide by deoxyhemoglobin vasodilates the human circulation. Nat Med 9, 1498-1505, doi:10.1038/nm954 (2003).
26 Gladwin, M. T., Raat, N. J. H., Shiva, S., Dezfulian, C., Hogg, N., Kim-Shapiro, D. B. & Patel, R. P. Nitrite as a vascular endocrine nitric oxide reservoir that contributes to hypoxic signaling, cytoprotection, and vasodilation. Am. J. Physiol.—Heart Circul. Physiol. 291, H2026-H2035 (2006).
27 Kim-Shapiro, D. B. & Gladwin, M. T. Mechanisms of nitrite bioactivation. Nitric Oxide 38, 58-68, doi:10.1016/j.niox.2013.11.002 (2014). PMC3999231
28 Gladwin, M. T. & Kim-Shapiro, D. B. The functional nitrite reductase activity of the heme-globins. Blood 112, 2636-2647, doi:10.1182/blood-2008-01-115261 (2008). Pmc2583304
29 Brooks, J. The action of nitrite on Haemoglobin in the absence of oxygen. Proc. Royal Soc. London—Series B, Biol. Sci. 123, 368-382 (1937).
30 Huang, K. T., Keszler, A., Patel, N., Patel, R. P., Gladwin, M. T., Kim-Shapiro, D. B. & Hogg, N. The Reaction between Nitrite and Deoxyhemoglobin: Reassessment of the reaction kinetics and stoichiometry. J. Biol. Chem. 280, 31126-31131 (2005).
31 Jeffers, A., Xu, X. L., Huang, K. T., Cho, M., Hogg, N., Patel, R. P. & Kim-Shapiro, D. B. Hemoglobin mediated nitrite activation of soluble guanylyl cyclase. Comp. Biochem. Physiol. A—Mol. Integr. Physiol. 142, 130-135 (2005).
32 Doyle, M. P. & Hoekstra, J. W. Oxidation of Nitrogen-Oxides by Bound Dioxygen in Hemoproteins. J Inorg Biochem 14, 351-358 (1981).
33 Eich, R. F., Li, T. S., Lemon, D. D., Doherty, D. H., Curry, S. R., Aitken, J. F., Mathews, A. J., Johnson, K. A., Smith, R. D., Phillips, G. N. & Olson, J. S. Mechanism of NO-induced oxidation of myoglobin and hemoglobin. Biochemistry—US 35, 6976-6983 (1996).
34 Herold, S., Exner, M. & Nauser, T. Kinetic and mechanistic studies of the NO center dot-mediated oxidation of oxymyoglobin and oxyhemoglobin. Biochemistry—US 40, 3385-3395 (2001).
35 Sharma, V. S. & Ranney, H. M. Dissociation of NO from Nitrosylhemoglobin. J. Biol. Chem. 253, 6467-6472 (1978).
36 Azizi, F., Shields, H., Yakubu, M., King, S. B. & Kim-Shapiro, D. B. Measurements of the rate of hemoglobin denitrosylation. Biophys. J. 84, 33a-33a (2003).
37 Cooper, C. E. Nitric oxide and iron proteins. Biochim. Biophys. Acta-Bioenerg. 1411, 290-309 (1999).
38 Moore, E. G. & Gibson, Q. H. Cooperativity in Dissociation of Nitric-Oxide from Hemoglobin. J. Biol. Chem. 251, 2788-2794 (1976).
39 Sun, C. W., Yang, J. N., Kleschyov, A. L., Zhuge, Z. B., Carlstrom, M., Pernow, J., Wajih, N., Isbell, T. S., Oh, J. Y., Cabrales, P., Tsai, A. G., Townes, T., Kim-Shapiro, D. B., Patel, R. P. & Lundberg, J. O. Hemoglobin beta 93 Cysteine Is Not Required for Export of Nitric Oxide Bioactivity From the Red Blood Cell. Circulation 139, 2654-2663, doi:10.1161/circulationaha.118.039284 (2019).
40 Radomski, M. W., Palmer, R. M. J. & Moncada, S. COMPARATIVE PHARMACOLOGY OF ENDOTHELIUM-DERIVED RELAXING FACTOR, NITRIC-OXIDE AND PROSTACYCLIN IN PLATELETS. Brit J Pharmacol 92, 181-187
41 Schafer, A., Wiesmann, F., Neubauer, S., Eigenthaler, M., Bauersachs, J. & Channon, K. Rapid Regulation of Platelet Activation In Vivo by Nitric Oxide. Circulation 109, 1819-1822 (2004).
42 Srihirun, S., Piknova, B., Sibmooh, N. & Schechter, A. N. Phosphorylated vasodilator-stimulated phosphoprotein (P-VASP(Ser239)) in platelets is increased by nitrite and partially deoxygenated erythrocytes. Plos One 13, doi:10.1371/journal.pone.0193747 (2018).
43 Lundberg, J. O., Weitzberg, E. & Gladwin, M. T. The nitrate-nitrite-nitric oxide pathway in physiology and therapeutics. Nat Rev Drug Discov 7, 156-167, doi:10.1038/nrd2466 (2008).
44 Presley, T. D., Morgan, A. R., Bechtold, E., Clodfelter, W., Dove, R. W., Jennings, J. M., Kraft, R. A., King, S. B., Laurienti, P. J., Rejeski, W. J., Burdette, J. H., Kim-Shapiro, D. B. & Miller, G. D. Acute effect of a high nitrate diet on brain perfusion in older adults. Nitric Oxide 24, 34-42, doi:10.1016/j.niox.2010.10.002 (2011). PMC3018552
45 Petrie, M., Rejeski, W. J., Basu, S., Laurienti, P. J., Marsh, A. P., Norris, J. L., Kim-Shapiro, D. B. & Burdette, J. H. Beet Root Juice: An Ergogenic Aid for Exercise and the Aging Brain. The journals of gerontology. Series A, Biological sciences and medical sciences 72, 1284-1289, doi:10.1093/gerona/glw219 (2017).
46 Eggebeen, J., Kim-Shapiro, D. B., Haykowsky, M., Morgan, T. M., Basu, S., Brubaker, P., Rejeski, J. & Kitzman, D. W. One Week of Daily Dosing With Beetroot Juice Improves Submaximal Endurance and Blood Pressure in Older Patients With Heart Failure and Preserved Ejection Fraction. JACC Heart Fail 4, 428-437, doi:10.1016/j.jchf2015.12.013 (2016). PMC4892939
47 Kapil, V., Khambata, R. S., Robertson, A., Caulfield, M. J. & Ahluwalia, A. Dietary Nitrate Provides Sustained Blood Pressure Lowering in Hypertensive Patients A Randomized, Phase 2, Double-Blind, Placebo-Controlled Study. Hypertension 65, 320-U174, doi:10.1161/hypertensionaha.114.04675 (2015).
48 Velmurugan, S., Kapil, V., Ghosh, S. M., Davies, S., McKnight, A., Aboud, Z., Khambata, R. S., Webb, A. J., Poole, A. & Ahluwalia, A. Antiplatelet effects of dietary nitrate in healthy volunteers: Involvement of cGMP and influence of sex (vol 65, pg 1521, 2013). Free Radic. Biol. Med. 84, 385-385, doi:10.1016/j.freeradbiomed.2015.03.015 (2015).
49 Jones, D. A., Pellaton, C., Velmurugan, S., Rathod, K. S., Andiapen, M., Antoniou, S., van Eijl, S., Webb, A. J., Westwood, M. A., Parmar, M. K., Mathur, A. & Ahluwalia, A. Randomized Phase 2 Trial of Intracoronary Nitrite During Acute Myocardial Infarction. Circ. Res. 116, 437-+, doi:10.1161/circresaha.116.305082 (2015).
50 Borlaug, B. A., Melenovsky, V. & Koepp, K. E. Inhaled Sodium Nitrite Improves Rest and Exercise Hemodynamics in Heart Failure With Preserved Ejection Fraction. Circ. Res. 119, 880-886, doi:10.1161/circresaha.116.309184 (2016).
51 Nagababu, E., Ramasamy, S., Abernethy, D. R. & Rifkind, J. M. Active nitric oxide produced in the red cell under hypoxic conditions by deoxyhemoglobin-mediated nitrite reduction. J. Biol. Chem. 278, 46349-46356 (2003).
52 Nagababu, E., Ramasamy, S. & Rifkind, J. M. Intermediates detected by visible Spectroscopy during the reaction of nitrite with deoxyhemoglobin: The effect of nitrite concentration and diphosphoglycerate. Biochemistry—US 46, 11650-11659, doi:10.1021/bi700364e (2007).
53 Basu, S., Grubina, R., Huang, J., Conradie, J., Huang, Z., Jeffers, A., Jiang, A., He, X., Azarov, I., Seibert, R., Mehta, A., Patel, R., King, S. B., Hogg, N., Ghosh, A., Gladwin, M. T. & Kim-Shapiro, D. B. Catalytic generation of N2O3 by the concerted nitrite reductase and anhydrase activity of hemoglobin. Nature chemical biology 3, 785-794, doi:10.1038/nchembio.2007.46 (2007).
54 Bunn, H. F. & Jandl, J. H. EXCHANGE OF HEME AMONG HEMOGLOBINS AND BETWEEN HEMOGLOBIN AND ALBUMIN. J. Biol. Chem. 243, 465-& (1968).
55 Nishikura, K. Effect of Inositol Hexaphosphate on Haem Exchange in Aquomet, Fluoromet and Nitrosyl Haemoglobins. J. Mol. Biol. 111, 91-93 (1977).
56 Hille, R., Olson, J. S. & Palmer, G. Spectral Transitions of Nitrosyl Hemes During Ligand-Binding to Hemoglobin. J. Biol. Chem. 254, 2110-2120 (1979).
57 Ignarro, L. J., Adams, J. B., Horwitz, P. M. & Wood, K. S. Activation of Soluble Guanylate-Cyclase by No-Hemoproteins Involves No-Heme Exchange—Comparison of Heme-Containing and Heme-Deficient Enzyme Forms. J. Biol. Chem. 261, 4997-5002 (1986).
58 Kleschyov, A. L. The NO-heme signaling hypothesis. Free Radic. Biol. Med. 112, 544-552, doi:10.1016/j.freeradbiomed.2017.08.025 (2017).
59 Desmet, K. D., Paz, D. A., Corry, J. J., Eells, J. T., Wong-Riley, M. T. T., Henry, M. M., Buchmann, E. V., Connelly, M. P., Dovi, J. V., Liang, H. L., Henshel, D. S., Yeager, R. L., Millsap, D. S., Lim, J., Gould, L. J., Das, R., Jett, M., Hodgson, B. D., Margolis, D. & Whelan, H. T. Clinical and experimental applications of NIR-LED photobiomodulation. Photomedicine and Laser Surgery 24, 121-128, doi:10.1089/pho.2006.24.121 (2006).
60 Huang, Y. Y., Sharma, S. K., Carroll, J. & Hamblin, M. R. BIPHASIC DOSE RESPONSE IN LOW LEVEL LIGHT THERAPY—AN UPDATE. Dose-Response 9, 602-618, doi:10.2203/dose-response.11-009.Hamblin (2011).
61 Uozumi, Y., Nawashiro, H., Sato, S., Kawauchi, S., Shima, K. & Kikuchi, M. Targeted Increase in Cerebral Blood Flow by Transcranial Near-Infrared Laser Irradiation. Lasers in Surgery and Medicine 42, 566-576, doi:10.1002/lsm.20938 (2010).
62 Blanco, N. J., Maddox, W. T. & Gonzalez-Lima, F. Improving executive function using transcranial infrared laser stimulation. Journal of Neuropsychology 11, 14-25, doi:10.1111/jnp.12074 (2017).
63 Wang, X. L., Tian, F. H., Soni, S. S., Gonzalez-Lima, F. & Liu, H. L. Interplay between up-regulation of cytochrome-c-oxidase and hemoglobin oxygenation induced by near-infrared laser. Scientific Reports 6, doi:10.1038/srep30540 (2016).
64 Karu, T. I., Pyatibrat, L. V., Kolyakov, S. F. & Afanasyeva, N. I. Absorption measurements of a cell monolayer relevant to phototherapy: Reduction of cytochrome c oxidase under near IR radiation. J. Photochem. Photobiol. B-Biol. 81, 98-106, doi:10.1016/j.jphotobiol.2005.07.002 (2005).
65 Wong-Riley, M. T. T., Liang, H. L., Eells, J. T., Chance, B., Henry, M. M., Buchmann, E., Kane, M. & Whelan, H. T. Photobiomodulation directly benefits primary neurons functionally inactivated by toxins—Role of cytochrome c oxidase. J. Biol. Chem. 280, 4761-4771, doi:10.1074/jbc.M409650200 (2005).
66 Lohr, N. L., Keszler, A., Pratt, P., Bienengraber, M., Warltier, D. C. & Hogg, N. Enhancement of nitric oxide release from nitrosyl hemoglobin and nitrosyl myoglobin by red/near infrared radiation: Potential role in cardioprotection. Journal of Molecular and Cellular Cardiology 47, 256-263, doi:10.1016/j.yjmcc.2009.03.009 (2009).
67 Keszler, A., Lindemer, B., Weihrauch, D., Jones, D., Hogg, N. & Lohr, N. L. Red/near infrared light stimulates release of an endothelium dependent vasodilator and rescues vascular dysfunction in a diabetes model. Free Radic. Biol. Med. 113, 157-164, doi:https://doi.org/10.1016/j.freeradbiomed.2017.09.012 (2017).
68 Jailer, I. H., Fredenburgh, J. C., Hirsh, J. & Weitz, J. I. Medical device-induced thrombosis: what causes it and how can we prevent it? Journal of Thrombosis and Haemostasis 13, S72-S81, doi:10.1111/jth.12961 (2015).
69 Koliopoulou, A., McKellar, S. H., Rondina, M. & Selzman, C. H. Bleeding and thrombosis in chronic ventricular assist device therapy: focus on platelets. Current Opinion in Cardiology 31, 299-307, doi:10.1097/hco.0000000000000284 (2016).
70 Meyer, A. D., Rishmawi, A. R., Kamucheka, R., Lafleur, C., Batchinsky, A. I., Mackman, N. & Cap, A. P. Effect of blood flow on platelets, leukocytes, and extracellular vesicles in thrombosis of simulated neonatal extracorporeal circulation. Journal of Thrombosis and Haemostasis, doi:10.1111/jth.14661.
71 Goudie, M. J., Pant, J. & Handa, H. Liquid-infused nitric oxide-releasing (LINORel) silicone for decreased fouling, thrombosis, and infection of medical devices. Scientific Reports 7, doi:10.1038/s41598-017-14012-9 (2017).
72 Ignarro, L. J., Byrns, R. E., Buga, G. M. & Wood, K. S. Endothelium-Derived Relaxing Factor from Pulmonary-Artery and Vein Possesses Pharmacological and Chemical-Properties Identical to Those of Nitric-Oxide Radical. Circ. Res. 61, 866-879 (1987).
73 Palmer, R. M. J., Ferrige, A. G. & Moncada, S. Nitric-Oxide Release Accounts for the Biological-Activity of Endothelium-Derived Relaxing Factor. Nature 327, 524-526 (1987).
74 Furchgott, R. F. in Vasodilatation: Vascular Smooth Muscle, Peptides, Autonomic Nerves, and Endothelium (ed P. M. Vanhoutte) 401-414 (Raven Press, 1988).
75 Ignarro, L. J., Byrns, R. E., and Wood, K. S. in Vasodilatation: Vascular Smooth Muscle, Peptides, Autonomic Nerves, and Endothelium (ed P. M. Vanhoutte) 427-435 (Raven Press, 1988).
76 Roberts, W., Michno, A., Aburima, A. & Naseem, K. M. Nitric oxide inhibits von Willebrand factor-mediated platelet adhesion and spreading through regulation of integrin alpha(IIb)beta(3) and myosin light chain. Journal of Thrombosis and Haemostasis 7, 2106-2115, doi:10.1111/j.1538-7836.2009.03619.x (2009).
77 Adams, M. R., Jessup, W., Hailstones, D. & Celermajer, D. S. L-arginine reduces human monocyte adhesion to vascular endothelium and endothelial expression of cell adhesion molecules. Circulation 95, 662-668 (1997).
78 Jin, H.-B., Yang, Y.-B., Song, Y.-L., Zhang, Y.-c. & Li, Y.-R. Lipoic acid attenuates the expression of adhesion molecules by increasing endothelial nitric-oxide synthase activity. Molecular Biology Reports 40, 377-382, doi:10.1007/s11033-012-2071-4 (2013).
79 Kubes, P., Suzuki, M. & Granger, D. N. Nitric oxide: an endogenous modulator of leukocyte adhesion. Proc Natl Acad Sci USA 88, 4651-4655 (1991).
80 Radomski, M. W., Rees, D. D., Dutra, A. & Moncada, S. S-NITROSO-GLUTATHIONE INHIBITS PLATELET ACTIVATION INVITRO AND INVIVO. Brit J Pharmacol 107, 745-749 (1992).
81 Simon, D. I., Stamler, J. S., Jaraki, O., Keaney, J. F., Osborne, J. A., Francis, S. A., Singel, D. J. & Loscalzo, J. Antiplatelet Properties of Protein S-Nitrosothiols Derived from Nitric-Oxide and Endothelium-Derived Relaxing Factor. Arterioscler. Thromb. 13, 791-799 (1993).
82 Helms, C. C., Kapadia, S., Gilmore, A. C., Lu, Z. X., Basu, S. & Kim-Shapiro, D. B. Exposure of fibrinogen and thrombin to nitric oxide donor ProliNONOate affects fibrin clot properties. Blood Coagul. Fibrinolysis 28, 356-364, doi:10.1097/mbc.0000000000000602 (2017).
83 Karandashov, V. I., Petukhov, E. B. & Zrodnikov, V. S. Changes in platelet aggregability after blood irradiation by helium-neon laser and red light emitting diodes. B Exp Biol Med+126, 1215-1217, doi:10.1007/bf02447155 (1998).
84 Spasov, A. A., Nedogoda, V. V. & Konan, K. Mechanism of hypocoagulative effect of low-intensity laser radiation. B Exp Biol Med+ 126, 672-674, doi:10.1007/bf02446056 (1998).
85 Brill, A. G., Brill, G. E., Kirichuk, V. F., Shenkman, B., Tamarin, I., Dardik, R., Varon, D. & Savion, N. Effect of He—Ne laser on platelet activation and aggregation. B Exp Biol Med+128, 700-701, doi:10.1007/bf02433318 (1999).
86 Brill, A. G., Shenkman, B., Brill, G. E., Tamarin, I., Dardik, R., Kirichuk, V. F., Savion, N. & Varon, D. Blood irradiation by He—Ne laser induces a decrease in platelet responses to physiological agonists and an increase in platelet cyclic GMP. Platelets 11, 87-93 (2000).
87 Rola, P., Doroszko, A., Szahidewicz-Krupska, E., Rola, P., Dobrowolski, P., Skomro, R., Szymczyszyn, A., Mazur, G. & Derkacz, A. Low-Level Laser Irradiation Exerts Antiaggregative Effect on Human Platelets Independently on the Nitric Oxide Metabolism and Release of Platelet Activation Markers. Oxidative Medicine and Cellular Longevity 2017, doi:10.1155/2017/6201797 (2017).
88 Almeida, C. B., Botelho Souza, L. E., Leonardo, F. C., Maranhao Costa, F. T., Werneck, C. C., Covas, D. T., Costa, F. F. & Conran, N. Acute hemolytic vascular inflammatory processes are prevented by nitric oxide replacement or a single dose of hydroxyurea. Blood 126, 711-720, doi:10.1182/blood-2014-12-616250 (2015).
89 Maley, J. H., Lasker, G. F. & Kadowitz, P. J. Nitric oxide and disorders of the erythrocyte: emerging roles and therapeutic targets. Cardiovascular & hematological disorders drug targets 10, 284-291 (2010).
90 Eisenhut, M. Severe Hemolysis as a Potential Contributing Factor in the Pathophysiology of Cerebral Malaria. Clinical Infectious Diseases 60, 1138-1138, doi:10.1093/cid/ciu1149 (2015).
91 Donadee, C., Raat, N. J. H., Kanias, T., Tejero, J., Lee, J. S., Kelley, E. E., Zhao, X. J., Liu, C., Reynolds, H., Azarov, I., Frizzell, S., Meyer, E. M., Donnenberg, A. D., Qu, L. R., Triulzi, D., Kim-Shapiro, D. B. & Gladwin, M. T. Nitric Oxide Scavenging by Red Blood Cell Microparticles and Cell-Free Hemoglobin as a Mechanism for the Red Cell Storage Lesion. Circulation 124, 465-U294, doi:10.1161/Circulationaha.110.008698 (2011).
92 Gladwin, M. T. & Kim-Shapiro, D. B. Storage lesion in banked blood due to hemolysis-dependent disruption of nitric oxide homeostasis. Curr. Opin. Hematol. 16, 515-523, doi:10.1097/MOH.0b013e32833157f4 (2009).
93 Hirai, D. M., Musch, T. I. & Poole, D. C. Exercise training in chronic heart failure: improving skeletal muscle O2 transport and utilization. American Journal of Physiology—Heart and Circulatory Physiology, doi:10.1152/ajpheart.00469.2015 (2015).
94 Bertoluci, M. C., Ce, G. V., da Silva, A. M., Wainstein, M. V., Boff, W. & Punales, M. Endothelial dysfunction as a predictor of cardiovascular disease in type 1 diabetes. World journal of diabetes 6, 679-692, doi:10.4239/wjd.v6.i5.679 (2015).
95 Li, Q., Youn, J. Y. & Cai, H. Mechanisms and consequences of endothelial nitric oxide synthase dysfunction in hypertension. Journal of Hypertension 33, 1128-1136, doi:10.1097/hjh.0000000000000587 (2015).
96 Rajapakse, N. W., Head, G. A. & Kaye, D. M. Say NO to Obesity-Related Hypertension Role of the L-Arginine-Nitric Oxide Pathway. Hypertension 67, 813-819, doi:10.1161/hypertensionaha.116.06778 (2016).
97 Rajagopalan, S., Al-Shaheen, A., Mackay, I., Bachoo, P. & Brittenden, J. Nitric oxide bioavailability decreases with severity of peripheral artery disease. British Journal of Surgery 99, 2-2 (2012).
98 Bauersachs, J. & Widder, J. D. Endothelial dysfunction in heart failure. Pharmacological Reports 60, 119-126 (2008).
99 Marti, C. N., Gheorghiade, M., Kalogeropoulos, A. P., Georgiopoulou, V. V., Quyyumi, A. A. & Butler, J. Endothelial Dysfunction, Arterial Stiffness, and Heart Failure. J Am Coll Cardiol 60, 1455-1469, doi:10.1016/j.jacc.2011.11.082 (2012).
100 Sharma, A., Sellers, S., Stefanovic, N., Leung, C., Tan, S. M., Huet, O., Granville, D. J., Cooper, M. E., de Haan, J. B. & Bernatchez, P. Direct Endothelial Nitric Oxide Synthase Activation Provides Atheroprotection in Diabetes-Accelerated Atherosclerosis. Diabetes 64, 3937-3950, doi:10.2337/db15-0472 (2015).
101 Aggarwal, A., Madigan, M. C., Cody, E. & Tzeng, E. A Role for Topical Nitrite in Diabetic Wound Healing. J Am Coll Surgeons 221, S176-S176, doi:10.1016/j.jamcollsurg.2015.07.418 (2015).
102 Minniti, C. P., Gorbach, A. M., Xu, D. H., Hon, Y. Y., Delaney, K. M., Seidel, M., Malik, N., Peters-Lawrence, M., Cantilena, C., Nichols, J. S., Mendelsohn, L., Conrey, A., Grimes, G. & Kato, G. J. Topical sodium nitrite for chronic leg ulcers in patients with sickle cell anaemia: a phase 1 dose-finding safety and tolerability trial. Lancet Haematology 1, E95-E103, doi:10.1016/s2352-3026(14)00019-2 (2014).
103 Gutsaeva, D. R., Montero-Huerta, P., Parkerson, J. B., Yerigenahally, S. D., Ikuta, T. & Head, C. A. Molecular mechanisms underlying synergistic adhesion of sickle red blood cells by hypoxia and low nitric oxide bioavailability. Blood 123, 1917-1926, doi:10.1182/blood-2013-06-510180 (2014).
104 Marsch, E., Sluimer, J. C. & Daemen, M. Hypoxia in atherosclerosis and inflammation. Curr. Opin. Lipidology 24, 393-400, doi:10.1097/MOL.0b013e32836484a4 (2013).
105 Bovill, E. G. & van der Vliet, A. Venous Valvular Stasis-Associated Hypoxia and Thrombosis: What Is the Link? Annual Review of Physiology, Vol 73 73, 527-545, doi:10.1146/annurev-physiol-012110-142305 (2011).
106 Bento, C. F. & Pereira, P. Regulation of hypoxia-inducible factor 1 and the loss of the cellular response to hypoxia in diabetes. Diabetologia 54, 1946-1956, doi:10.1007/s00125-011-2191-8 (2011).
107 Kim-Shapiro, D. B., Schechter, A. N. & Gladwin, M. T. Unraveling the Reactions of Nitric Oxide, Nitrite, and Hemoglobin in Physiology and Therapeutics. Arterioscler Thromb Vasc Biol 26, 697-705 (2006).
108 Lundberg, J. O. et al. Nitrate and nitrite in biology, nutrition and therapeutics. Nat Chem Biol 5, 865-869, doi:10.1038/nchembio.260 (2009). PMC4038383
109 van Faassen, E. E., Bahrami, S., Feelisch, M., Hogg, N., Kelm, M., Kim-Shapiro, D. B., Kozlov, A. V., Li, H., Lundberg, J. O., Mason, R., Nohl, H., Rassaf, T., Samouilov, A., Slama-Schwok, A., Shiva, S., Vanin, A. F., Weitzberg, E., Zweier, J. & Gladwin, M. T. Nitrite as regulator of hypoxic signaling in mammalian physiology. Med Res Rev 29, 683-741, doi:10.1002/med.20151 (2009). PMC2725214
110 Patel, R. P., Hogg, N. & Kim-Shapiro, D. B. The potential role of the red blood cell in nitrite-dependent regulation of blood flow. Cardiovasc Res 89, 507-515, doi:10.1093/cvr/cvq323 (2011). PMC3028972
111 Basu, S., Wang, X., Gladwin, M. T. & Kim-Shapiro, D. B. Chemiluminescent Detection of S-Nitrosated Proteins: Comparison of Tri-iodide, Copper/CO/Cysteine, and Modified Copper/Cysteine Methods. Methods Enzymol. 440, 137-156, doi:10.1016/s0076-6879(07)00808-7 (2008).
112 Gladwin, M. T. Chemiluminescent detection of S-nitrosated proteins: comparison of tri-iodide, copper/CO/cysteine, and modified copper/cysteine methods. The Journal of biological chemistry 440, 137-156, doi:10.1074/jbc.M705222200 10.1016/s0076-6879(07)00808-7 (2008).
113 Xu, X. L., Cho, M., Spencer, N. Y., Patel, N., Huang, Z., Shields, H., King, S. B., Gladwin, M. T., Hogg, N. & Kim-Shapiro, D. B. Measurements of nitric oxide on the heme iron and beta-93 thiol of human hemoglobin during cycles of oxygenation and deoxygenation. Proc. Natl. Acad. Sci. U S. A. 100, 11303-11308 (2003).
114 Grubina, R., Basu, S., Tiso, M., Kim-Shapiro, D. B. & Gladwin, M. T. Nitrite reductase activity of hemoglobin S (sickle) provides insight into contributions of heme redox potential versus ligand affinity. The Journal of biological chemistry 283, 3628-3638, doi:10.1074/jbc.M705222200 (2008).
115 Basu, S., Keszler, A., Azarova, N. A., Nwanze, N., Perlegas, A., Shiva, S., Broniowska, K. A., Hogg, N. & Kim-Shapiro, D. B. A novel role for cytochrome c: Efficient catalysis of S-nitrosothiol formation. Free Radic. Biol. Med. 48, 255-263, doi:10.1016/j.freeradbiomed.2009.10.049 (2010). Pmc2819623
116 Azarov, I., Huang, K. T., Basu, S., Gladwin, M. T., Hogg, N. & Kim-Shapiro, D. B. Nitric oxide scavenging by red blood cells as a function of hematocrit and oxygenation. J. Biol. Chem. 280, 39024-38032 (2005).
117 Tejero, J., Basu, S., Helms, C., Hogg, N., King, S. B., Kim-Shapiro, D. B. & Gladwin, M.
T. Low NO Concentration Dependence of Reductive Nitrosylation Reaction of Hemoglobin. Journal of Biological Chemistry 287, 18262-18274, doi:10.1074/jbc.M111.298927 (2012). Pmc3600400
118 Corti, P., Xue, J., Tejero, J., Wajih, N., Sun, M., Stolz, D. B., Tsang, M., Kim-Shapiro, D. B. & Gladwin, M. T. Globin X is a six-coordinate globin that reduces nitrite to nitric oxide in fish red blood cells. Proc Natl Acad Sci USA 113, 8538-8543, doi:10.1073/pnas.1522670113 (2016). PMC4968712
119 Sato, H., Zhao, Z. Q., Jordan, J. E., Todd, J. C., Riley, R. D., Taft, C. S., Hammon, J. W., Li, P., Ma, X. L. & VintenJohansen, J. Basal nitric oxide expresses endogenous cardioprotection during reperfusion by inhibition of neutrophil-mediated damage after surgical revascularization. J Thorac Cardiov Sur 113, 399-409, doi:10.1016/s0022-5223(97)70338-6 (1997).
120 Lee, D. J., Aboushwareb, T., Steen, J., Berry, J., Kon, N. D., Atala, A., Kincaid, E. H., Jordan, J. E. & Yoo, J. J. Reendothelialization of tissue engineered heart valves using endothelial progenitor cells from valvular disease patients: A feasibility study. J Am Coll Surgeons 209, S92-S92, doi:10.1016/j.jamcollsurg.2009.06.228 (2009).
121 Argenta, L. C., Morykwas, M. J., Mays, J. J., Thompson, E. A., Hammon, J. W. & Jordan, J. E. Reduction of Myocardial Ischemia-Reperfusion Injury by Mechanical Tissue Resuscitation Using Sub-Atmospheric Pressure. Journal of Cardiac Surgery 25, 247-252, doi:10.1111/j.1540-8191.2009.00972.x (2010).
122 Berry, J. L., Steen, J. A., Williams, J. K., Jordan, J. E., Atala, A. & Yoo, J. J. Bioreactors for Development of Tissue Engineered Heart Valves. Ann Biomed Eng 38, 3272-3279, doi:10.1007/s10439-010-0148-6 (2010).
123 Jordan, J. E., Pereira, B. D., Lane, M. R., Morykwas, M. J., McGee, M. & Argenta, L. C. Mechanical Tissue Resuscitation (MTR): A Nonpharmacological Approach to Treatment of Acute Myocardial Infarction. Journal of Cardiac Surgery 30, 659-667, doi:10.1111/jocs.12580 (2015).
124 Schmidt, B. S., Jordan, J. E., Lane, M. R., DiPasquale, V. M., Graf, L. P., Ootaki, Y. & Ungerleider, R. M. Timing of adding blood to prime affects inflammatory response to neonatal cardiopulmonary bypass. Cardiology in the Young 27, 480-487, doi:10.1017/s1047951116000767 (2017).
125 Rahbar, E., Mori, D. & Moore, J. E. Three-dimensional Analysis of Flow Disturbances Caused by Clots in Inferior Vena Cava Filters. Journal of Vascular and Interventional Radiology 22, 835-842, doi:10.1016/j.jvir.2010.12.024 (2011).
126 Cardenas, J. C., Rahbar, E., Pommerening, M. J., Baer, L. A., Matijevic, N., Cotton, B. A., Holcomb, J. B. & Wade, C. E. Measuring thrombin generation as a tool for predicting hemostatic potential and transfusion requirements following trauma. J. Trauma Acute Care Surg. 77, 839-845, doi:10.1097/ta.0000000000000348 (2014).
127 Pommerening, M. J., Rahbar, E., Minei, K., Holcomb, J. B., Wade, C. E., Schreiber, M. A., Cohen, M. J., Underwood, S. J., Nelson, M. & Cotton, B. A. Splenectomy is associated with hypercoagulable thrombelastography values and increased risk of thromboembolism. Surgery 158, 618-626, doi:10.1016/j.surg.2015.06.014 (2015).
128 Rahbar, E., Cardenas, J. C., Baimukanova, G., Usadi, B., Bruhn, R., Pati, S., Ostrowski, S. R., Johansson, P. I., Holcomb, J. B. & Wade, C. E. Endothelial glycocalyx shedding and vascular permeability in severely injured trauma patients. Journal of Translational Medicine 13, doi:10.1186/s12967-015-0481-5 (2015).
129 Rahbar, E., Cardenas, J. C., Matijevic, N., del Junco, D., Podbielski, J., Cohen, M. J., Cotton, B. A., Holcomb, J. B., Wade, C. E. & Early Whole Blood, I. TRAUMA, TIME, AND TRANSFUSIONS: A LONGITUDINAL ANALYSIS OF COAGULATION MARKERS IN SEVERELY INJURED TRAUMA PATIENTS RECEIVING MODIFIED WHOLE BLOOD OR COMPONENT BLOOD PRODUCTS. Shock 44, 417-425, doi:10.1097/shk.0000000000000449 (2015).
130 Robinson, B. R. H. et al. RISK FACTORS FOR THE DEVELOPMENT OF ACUTE RESPIRATORY DISTRESS SYNDROME FOLLOWING HEMORRHAGE. Shock 50, 258-264, doi:10.1097/shk.0000000000001073 (2018).
131 Cardenas, J. C. et al. TEG LYSIS SHUTDOWN REPRESENTS COAGULOPATHY IN BLEEDING TRAUMA PATIENTS: ANALYSIS OF THE PROPPR COHORT. Shock 51, 273-283, doi:10.1097/shk.0000000000001160 (2019).
132 Chang, R. et al. Earlier time to hemostasis is associated with decreased mortality and rate of complications: Results from the Pragmatic Randomized Optimal Platelet and Plasma Ratio trial. J. Trauma Acute Care Surg. 87, 342-349, doi:10.1097/ta.0000000000002263 (2019).
133 Hoshino, M., Maeda, M., Konishi, R., Seki, H. & Ford, P. C. Studies on the reaction mechanism for reductive nitrosylation of ferrihemoproteins in buffer solutions. J Am Chem Soc 118, 5702-5707 (1996).
134 Pereira, J. C. M., Iretskii, A. V., Han, R. M. & Ford, P. C. Dinitrosyl Iron Complexes with Cysteine. Kinetics Studies of the Formation and Reactions of DNICs in Aqueous Solution. J Am Chem Soc 137, 328-336, doi:10.1021/ja510393q (2015).
135 Truzzi, D. R., Augusto, 0. & Ford, P. C. Thiyl radicals are co-products of dinitrosyl iron complex (DNIC) formation. Chemical Communications 55, 9156-9159, doi:10.1039/c9cc04454j (2019).
136 Shipulina, N., Hunt, R. C., Shaklai, N. & Smith, A. Coordination of nitric oxide by heme-hemopexin. J Protein Chem 17, 255-260, doi:10.1023/a:1022536818947 (1998).
137 Yang, B. K., Vivas, E. X., Reiter, C. D. & Gladwin, M. T. Methodologies for the sensitive and specific measurement of S-nitrosothiols, iron-nitrosyls, and nitrite in biological samples. Free Radic. Res. 37, 1-10 (2003).
138 Smolenski, A. Novel roles of cAMP/cGMP-dependent signaling in platelets. Journal of Thrombosis and Haemostasis 10, 167-176, doi:10.1111/j.1538-7836.2011.04576.x (2012).
139 Estevez, B. & Du, X. P. New Concepts and Mechanisms of Platelet Activation Signaling. Physiology 32, 162-177, doi:10.1152/physiol.00020.2016 (2017).
140 Lindkvist, M., Fernberg, U., Ljungberg, L. U., Falker, K., Fernstrom, M., Hurtig-Wennlof, A. & Grenegard, M. Individual variations in platelet reactivity towards ADP, epinephrine, collagen and nitric oxide, and the association to arterial function in young, healthy adults. Thromb. Res. 174, 5-12, doi:10.1016/j.thromres.2018.12.008 (2019).
141 Irwin, C., Roberts, W. & Naseem, K. M. Nitric oxide inhibits platelet adhesion to collagen through cGMP-dependent and independent mechanisms: The potential role for S-nitrosylation. Platelets 20, 478-486, doi:10.3109/09537100903159375 (2009).
142 Riba, R., Sharifi, M., Farndale, R. & Naseem, K. M. Regulation of platelet guanylyl cyclase by collagen: evidence that Glycoprotein VI mediates platelet nitric oxide synthesis in response to collagen. Thromb. Haemost. 94, 395-403, doi:10.1160/th05-01-0027 (2005).
143 Wang, G. R., Zhu, Y., Halushka, P. V., Lincoln, T. M. & Mendelsohn, M. E. Mechanism of platelet inhibition by nitric oxide: In vivo phosphorylation of thromboxane receptor by cyclic GMP-dependent protein kinase. Proc. Natl. Acad. Sci. USA 95, 4888-4893, doi:10.1073/pnas.95.9.4888 (1998).
144 Longstaff, C. Measuring fibrinolysis: from research to routine diagnostic assays. Journal of Thrombosis and Haemostasis 16, 652-662, doi:10.1111/jth.13957 (2018).
145 Zhu, L., Cronin, T. & Narang, A. Wavelength-dependent photolysis of i-pentanal and t-pentanal from 280 to 330 nm. J Phys Chem A 103, 7248-7253, doi:10.1021/jp991540p (1999).
146 Park, J. W., Piknova, B., Nghiem, K., Lozier, J. N. & Schechter, A. N. Inhibitory effect of nitrite on coagulation processes demonstrated by thrombelastography. Nitric Oxide—Biol Ch 40, 45-51, doi:10.1016/j.niox.2014.05.006 (2014).
147 Joannidis, M. & Straaten, H. Clinical review: Patency of the circuit in continuous renal replacement therapy. Crit Care 11, doi:10.1186/cc5937 (2007).
148 Damodaran, V. B., Leszczak, V., Wold, K. A., Lantvit, S. M., Popat, K. C. & Reynolds, M. M. Antithrombogenic properties of a nitric oxide-releasing dextran derivative: evaluation of platelet activation and whole blood clotting kinetics. Rsc Advances 3, 24406-24414, doi:10.1039/c3ra45521a (2013).
149 Drohomirecka, A., Iwaszko, A., Walski, T., Pliszczak-Krol, A., Waz, G., Graczyk, S., Galecka, K., Czerski, A., Bujok, J. & Komorowska, M. Low-level light therapy reduces platelet destruction during extracorporeal circulation. Scientific Reports 8, doi:10.1038/s41598-018-35311-9 (2018).
150 Velmurugan, S., Kapil, V., Ghosh, S. M., Davies, S., McKnight, A., Aboud, Z., Khambata, R. S., Webb, A. J., Poole, A. & Ahluwalia, A. Antiplatelet effects of dietary nitrate in healthy volunteers: Involvement of cGMP and influence of sex. Free Radic. Biol. Med. 65, 1521-1532, doi:10.1016/j.freeradbiomed.2013.06.031 (2013).
151 Fealy, N., Aitkin, L., du Toit, E., Lo, S. & Baldwin, I. Faster Blood Flow Rate Does Not Improve Circuit Life in Continuous Renal Replacement Therapy: A Randomized Controlled Trial. Critcial Care Medicine 45, e1018-e1025 (2017).
152 Ng, M. S. Y., Tung, J. P. & Fraser, J. F. Platelet Storage Lesions: What More Do We Know Now? Transfusion Medicine Reviews 32, 144-154, doi:10.1016/j.tmrv.2018.04.001 (2018).
153 Chang, K. Y., Kim, S. H., Kim, Y. O., Jin, D. C., Song, H. C., Choi, E. J., Kim, Y. L., Kim, Y. S., Kang, S. W., Kim, N. H., Yang, C. W. & Kim, Y. K. The impact of blood flow rate during hemodialysis on all-cause mortality. Korean Journal of Internal Medicine 31, 1131-1139, doi:10.3904/kjim.2015.111 (2016).

What is claimed is:

1. A method for inhibiting device thrombosis for a patient, the method comprising:
circulating blood through an extracorporeal circuit coupled to the patient;
infusing nitrite into the blood such that the nitrite is bioactivated by red blood cells to form nitric oxide; and
illuminating the blood with far-red light to increase bioactivation of the nitrite by the red blood cells.

2. The method of claim 1, wherein infusing the nitrite into the blood comprises infusing the nitrite in line with the extracorporeal circuit.

3. The method of claim 1, wherein infusing the nitrite into the blood comprises continuously infusing the nitrite for a period of time while the blood circulates through the extracorporeal circuit.

4. The method of claim 1, wherein illuminating the blood with the far-red light comprises illuminating the blood with the far-red light in line with the extracorporeal circuit.

5. The method of claim 1, wherein illuminating the blood with the far-red light comprises continuously illuminating the blood with the far-red light for a period of time while the blood circulates through the extracorporeal circuit.

6. The method of claim 1, wherein illuminating the blood with the far-red light comprises passing the blood through a section of tubing disposed between a pair of light sources.

7. The method of claim 6, wherein the section of tubing is arranged in a coil.

8. The method of claim 6, wherein the pair of light sources comprise a pair of light-emitting diodes.

9. The method of claim 1, wherein illuminating the blood with the far-red light comprises illuminating the blood with the far-red light emitted at a wavelength of 660 nm.

10. The method of claim 1, further comprising maintaining the blood circulating through the extracorporeal circuit at a predetermined temperature using a circulating water bath.

11. A system for inhibiting device thrombosis for a patient, the system comprising:
an extracorporeal circuit configured to circulate blood of the patient;
a nitrite infusion device coupled to the extracorporeal circuit and configured to infuse nitrite into the blood such that the nitrite is bioactivated by red blood cells to form nitric oxide; and
one or more light sources disposed along the extracorporeal circuit and configured to illuminate the blood with far-red light to increase bioactivation of the nitrite by the red blood cells.

12. The system of claim 11, wherein the nitrite infusion device is configured to infuse the nitrite in line with the extracorporeal circuit.

13. The system of claim 11, wherein the nitrite infusion device is configured to infuse the nitrite for a period of time while the blood circulates through the extracorporeal circuit.

14. The system of claim 11, wherein the one or more light sources are configured to illuminate the blood with the far-red light in line with the extracorporeal circuit.

15. The system of claim 11, wherein the one or more light sources are configured to illuminate the blood with the far-red light for a period of time while the blood circulates through the extracorporeal circuit.

16. The system of claim 11, wherein the extracorporeal circuit comprises a section of tubing disposed adjacent to the one or more light sources.

17. The system of claim 16, wherein the section of tubing is arranged in a coil.

18. The system of claim 16, wherein the one or more light sources comprise a pair of light sources, and wherein the section of tubing is disposed between the pair of light sources.

19. The system of claim 18, wherein the pair of light sources comprise a pair of light-emitting diodes.

20. The system of claim 11, wherein the one or more light sources are configured to emit the far-red light at a wavelength of 660 nm.

21. The system of claim 11, further comprising a circulating water bath configured to maintain the blood circulating through the extracorporeal circuit at a predetermined temperature.
